(19) United States
(12) Patent Application Publication
(10) Pub. No.: US 2021/0129345 A1
(43) Pub. Date: May 6, 2021

(54) INTELLIGENT WHEERCHAIR SYSTEM HAVING MEDICAL MONITORING AND RESPONSE FUNCTION

(71) Applicant: SICHUAN GOLDEN RIDGE INTELLIGENCE SCIENCE & TECHNOLOGY CO., LTD., Chengdu, Sichuan (CN)

(72) Inventors: Weirong LIU, Beijing (CN); Jiaxin LI, Chengdu (CN); Yin JIAO, Gothenburg (SE); Li YAN, Gothenburg (SE); Dong DONG, Gothenburg (SE); Yifeng HUANG, Gothenburg (SE)

(73) Assignee: SICHUAN GOLDEN RIDGE INTELLIGENCE SCIENCE & TECHNOLOGY CO., LTD., Chengdu, Sichuan (CN)

(21) Appl. No.: 16/473,741

(22) PCT Filed: Jan. 22, 2017
(86) PCT No.: PCT/CN2017/072102

§ 371 Date: Jun. 26, 2019

(51) Int. Cl. B25J 11/00; A61B 5/021; A61B 5/024; A61B 5/145; A61B 5/029; A61B 5/08; A61B 5/0215; A61B 5/01; A61B 5/00

(52) U.S. Cl. CPC B25J 11/009; A61B 5/02108; A61B 5/024; A61B 5/14542; A61B 5/746; A61B 5/08; A61B 5/0215; A61B 5/01; A61B 5/6894; A61B 5/029

(57) ABSTRACT

A method and system for monitoring physiological information. The system comprises: sensors (230), wherein the sensors (230) comprise a motion sensor and a medical monitoring sensor, the motion sensor comprises a first type of sensor (1220) and a second type of sensor (1240), and the medical monitoring sensor is configured to acquire physiological information of a user; a motion assembly (920) executing a control parameter-based operation to move in the surroundings; a tripod head (930); and a processor (210) used to execute operations such as information receiving, map construction, route planning, and control parameter operation.

### TECHNICAL FIELD

The present disclosure generally relates to a system and method for medical monitoring, response function and control of an intelligent wheelchair, and in particular, to a mobile intelligent robot, an image detection and processing, a route search, and a control method for a robot movement.

### BACKGROUND

In daily life, a movable intelligent devices, such as a cleaning robot, an intelligent balance wheel and an intelligent wheelchair, is becoming more and more popular. The Intelligent wheelchair may combine a robot technology to assist people to walk. The Intelligent wheelchair may apply an intelligent robot system to implement some functions, such as a movement, sensing the surrounding, or a health monitoring. To provide a service in a specific region, the intelligent robot system may move automatically by identifying the surroundings according to a given map. With the rapid expansion of demand for this service, people may desire a multifunctional intelligent robot system that is able to update the map, plan a route and move automatically, in particular, an intelligent robot that may adapt to more complex surroundings.

The intelligent wheelchair may be usually used by the elder or people with a cognitive disorder or a movement disorder. It is desirable to develop the intelligent wheelchair that is able to monitor a user's physiological information and respond based on the physiological information.

### SUMMARY

An aspect of the present disclosure relates to a system for monitoring physiological information. The system may include sensors. The sensors may include a motion sensor and a medical monitoring sensor. The motion sensor may include a first type of sensor and a second type of sensor. The medical monitoring sensor may be configured to acquire physiological information of a user. The system may include a motion assembly. The system may include a tripod head and a processor in communication with a memory. When executing instructions, the processor may establish communication with the motion assembly and the tripod head via a communication port. The processor may acquire information from the sensors to construct a map. The processor may further plan a route based on the information, and generate control parameters based on the information.

Another aspect of the present disclosure relates to a method. The method may include establishing a communication between a motion assembly and a tripod head via a communication port. The method may include obtaining information from sensors of the motion assembly and the tripod head to construct a map. The method may also include planning a route based on the information, and generating control parameters based on the information.

Yet another aspect of the present disclosure relates to a non-transitory computer readable medium embodied as a computer program product. The computer program product may include a communication port for establishing a communication between a processor and a motion assembly, and a communication between the processor and a tripod head. The communication port may establish the communications by an application program interface (API).

### BRIEF DESCRIPTION OF THE DRAWINGS

The present disclosure is further described in terms of exemplary embodiments. These exemplary embodiments are described in detail with reference to the drawings. These embodiments are non-limiting exemplary embodiments, in which like reference numerals represent similar structures throughout the several views of the drawings.

FIG. 1 is a schematic diagram illustrating a robot control system for a scanning system according to some embodiments of the present disclosure;

FIG. 2 is a block diagram illustrating a robot in the robot control system illustrated in FIG. 1 according to some embodiments of the present disclosure;

FIG. 3 is a block diagram illustrating a processor in the robot illustrated in FIG. 2 according to some embodiments of the present disclosure;

FIG. 4 is a block diagram illustrating an analysis module in the processor illustrated in FIG. 3 according to some embodiments of the present disclosure;

FIG. 5 is a block diagram illustrating a navigation module in a processor according to some embodiments of the present disclosure;

FIG. 6 is a schematic diagram illustrating a motion control according to some embodiments of the present disclosure;

FIG. 7 is a schematic diagram illustrating a motion control according to some embodiments of the present disclosure;

FIG. 8 is a schematic diagram illustrating exemplary components of sensors illustrated in FIG. 2 according to some embodiments of the present disclosure;

FIG. 9 is a schematic diagram illustrating a body illustrated in FIG. 2 according to some embodiments of the present disclosure;

FIG. 10 is a schematic diagram illustrating a motion assembly according to some embodiments to the present disclosure;

FIG. 11 is a schematic diagram illustrating a structure of a tripod head illustrated in FIG. 9 according to some embodiments of the present disclosure;

FIG. 12 is a schematic diagram illustrating a robot system according to some embodiments of the present disclosure;

FIG. 13 is a flowchart illustrating an exemplary process for determining control parameters of a robot according to some embodiments of the present disclosure;

FIG. 14 is a flowchart illustrating an exemplary process for constructing a map according to some embodiments of the present disclosure;

FIG. 15 is a flowchart illustrating an exemplary process for determining one or more reference frames according to some embodiments of the present disclosure;

FIG. 16 is a flowchart illustrating an exemplary process for obtaining depth information, intensity information, and displacement information according to some embodiments of the present disclosure;

FIG. 17A is a flowchart illustrating an exemplary process for determining an initial value of a displacement according to some embodiments of the present disclosure;

FIG. 17B is a flowchart illustrating an exemplary process for determining a pose of a robot according to some embodiments of the present disclosure;

FIG. 18 is a schematic diagram illustrating an exemplary scheme for determining an included angle between a horizontal plane and a Z axis by a gyroscope and an accelerometer according to some embodiments of the present disclosure;

FIG. 19 is a flowchart illustrating an exemplary process for determining an angle corresponding to a reference frame according to some embodiments of the present disclosure; and

FIG. 20 is a flowchart illustrating an exemplary process for adjusting a motion of a sensor of an intelligent device in a vertical direction according to some embodiments of the present disclosure.

### DETAILED DESCRIPTION

In the detailed descriptions below, numerous specific details of the disclosure are set forth in order to provide a thorough understanding of the disclosure. However, it will be apparent to those skilled in the art that the present disclosure may be practiced without these details. In other instances, well-known methods, procedures, systems, components, and/or circuits in the present disclosure have been described at relatively high levels elsewhere and are not described in detail in this disclosure to avoid unnecessarily repeating.

It should be understood that the terms “system,” “device,” “unit,” and/or “module” are used in this disclosure to refer to a different component, component, portion, or component of the different levels of the order. However, if other expressions may achieve the same purpose, these terms may be replaced by other expressions.

It should be understood that when a device, a unit, or a module is referred to as “on”, “connected to”, or “coupled” to another device, unit, or module, it may be directly connected to, coupled to, or communicated with other devices, units or, modules on another device, unit, or module, or may be stored within the device, the unit, or the module, unless the context clearly indicates an exception. For example, the term “and/or” as used in this disclosure includes any and all combinations of one or more of the associated listed items.

The terminology used in the disclosure is only intended to describe a particular embodiment and is not intended to limit the scope of the disclosure. As used in the disclosure and the appended claims, the singular forms “a,” “an,” and “the” include plural referents unless the content clearly dictates otherwise. In general, the terms “include” and “comprise” are merely meant to include the features, integers, steps, operations, elements, and/or components that are specifically identified, and such expressions do not constitute an exclusive list, and other features, integers, steps, operations, elements, and/or components may be included.

These and other features, and characteristics of the present disclosure, as well as the methods of operations and functions of the related elements of structure and the combination of parts and economies of manufacture, may become more apparent upon consideration of the following description with reference to the accompanying drawings, all of which form part of this disclosure. It is to be expressly understood, however, that the drawings are for the purpose of illustration and description only and are not intended to limit the scope of the present disclosure. It is understood that the drawings are not to scale.

Moreover, the present disclosure describes only systems and methods for determining a status of an intelligent robot. It is understood that the description in this disclosure is merely one embodiment. The intelligent robot may also be applied to any type of an intelligent device or a vehicle other than the intelligent robot. For example, the systems or methods for the intelligent robot may be used in various intelligent device systems, such as a balance wheel, an unmanned ground vehicle (UGV), an intelligent wheelchair, or the like, or any combination thereof. The intelligent robot system may also be applied to any intelligent system including an application management and/or distribution, such as a system for sending and/or receiving express delivery and a system for carrying personnel or goods to certain locations.

The terms “robot,” “intelligent robot,” “intelligent device,” as used in this disclosure, may be used interchangeably to refer to an apparatus, a device, or a tool that may be moved and operated automatically. The term “user device” in this disclosure may refer to a tool that may be used to request a service, subscribe to a service, or facilitate the provision of a service. The term “mobile terminal” in this disclosure may refer to a tool or an interface that may be used by a user to control an intelligent robot.

With the acceleration of the aging society and the increase in the number of lower limb injuries caused by various diseases, work injuries, traffic accidents, etc., providing superior performance tools for the elder and the disabled has become one of the major concerns of the whole society. As a service robot, the intelligent wheelchair has various functions such as autonomous navigation, obstacle avoidance, human-machine dialogue, and provision of special services. It can provide a safe and convenient lifestyle for people with cognitive disabilities (e.g., the dementia patients), people with disabilities (e.g., the cerebral palsy, the quadriplegia, etc.) and the elder. It can improve their daily life quality and work quality, so as that they can regain an ability to take care of themselves, and reintegrate into society.

As a robotics application platform, the intelligent wheelchair combines various technologies in robotics field, for example, a robot navigation and positioning, a machine vision, a pattern recognition, a multi-sensor information fusion, and a human-machine interaction.

Intelligent wheelchairs may be categorized based on the navigation technology and the human-machine interface technology.

According to different human-machine interface technologies, the intelligent wheelchair may include a set human-machine interface-based intelligent wheelchair and a natural human-machine interface-based intelligent wheelchair. The set human-machine interface-based intelligent wheelchair may include but not limited to a joystick-controlled intelligent wheelchair, a button-controlled intelligent wheelchair, a steering wheel-controlled intelligent wheelchair, a touch-screen-controlled intelligent wheelchair, a menu-controlled intelligent wheelchair, or the like, or any combination thereof. The natural human-machine interface-based intelligent wheelchair may include but not limited to a voice-controlled intelligent wheelchair, a breath-controlled intelligent wheelchair, a head-controlled intelligent wheelchair, a gesture-controlled intelligent wheelchair, a tongue-action-controlled intelligent wheelchair, and a biosignal-controlled intelligent wheelchair, or the like, or any combination thereof. The biosignal-controlled intelligent wheelchair may include but not limited to an electroencephalogram (EEG) intelligent wheelchair, an electromyography (EMG) intelligent wheelchair, an electrooculogram (FOG) intelligent wheelchair, and so on.

According to different navigation technologies, the intelligent wheelchair may include a landmark navigation-based intelligent wheelchair, a map navigation-based intelligent wheelchair, a sensor navigation-based intelligent wheelchair, a visual navigation-based intelligent wheelchair, and so on. The sensor navigation-based intelligent wheelchair may include but not limited to an ultrasonic sensing type of intelligent wheelchair, an infrared sensing type of intelligent wheelchair, a laser ranging type of intelligent wheelchair, a collision sensing type of intelligent wheelchair, and so on.

In the present disclosure, an intelligent wheelchair system may use an intelligent robot to implement various functions, such as moving, changing directions, stopping, sensing the surroundings, drawing a map, or planning a route. It should be noted that the intelligent robot provided in this disclosure may also be used in other fields to achieve similar functions or purposes.

The positioning technologies used in the present disclosure may include a global positioning system (GPS) technology, a global navigation satellite system (GLONASS) technology, a compass navigation system (COMPASS) technology, a Galileo positioning system (Galileo) technology, a quasi-zenith satellite system (QZSS) technology, a wireless fidelity (WIFI) positioning technology, or the like, or any combination thereof. One or more of the positioning techniques may be used interchangeably in this disclosure.

The present disclosure describes an intelligent robot control system 100 as an exemplary system, and a method for constructing a map and planning a route for the intelligent robot control system 100. The method and the system of the present disclosure may be intended to construct a map according to, for example, information obtained by the intelligent robot control system 100. The obtained information may be acquired by a sensor(s) located in the intelligent robot control system 100. The sensor(s) may be optical or electromagnetic. For example, the sensor may include a camera or a laser radar.

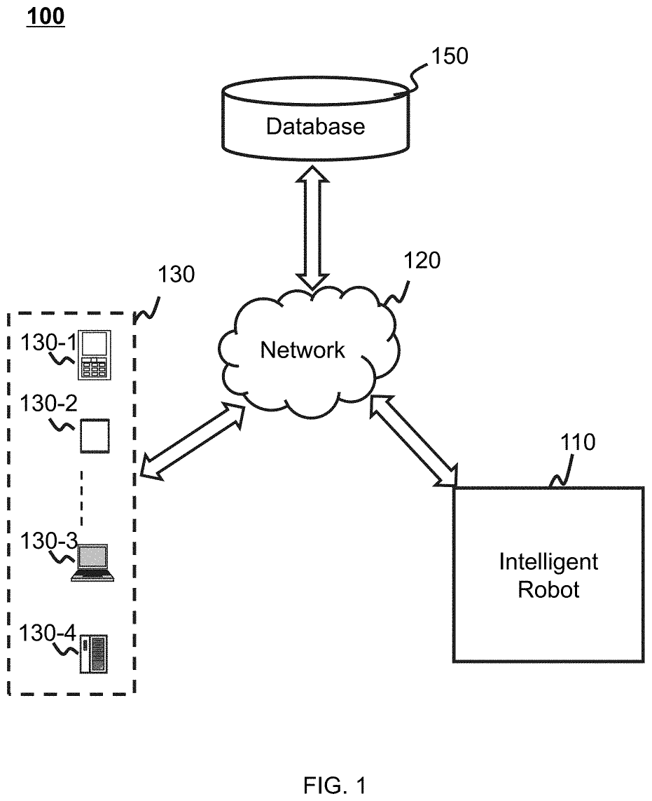

FIG. 1 is a schematic diagram illustrating an exemplary intelligent robot control system 100 according to some embodiments of the present disclosure. The intelligent robot control system 100 may include an intelligent robot 110, a network 120, a user device 130, and a database 140. A user may use the user device 130 to control the intelligent robot through the network 120.

The intelligent robot 110 may establish a communication with the user device 130. The communication between the intelligent robot 110 and the user device 130 may be wired or wireless. For example, the intelligent robot 110 may establish the communication with the user device 130 or the database 140 via the network 120, and the intelligent robot 110 may be wirelessly controlled based on an operational command (e.g., a command for moving or rotating) from the user device 130. As another example, the intelligent robot 110 may be directly connected to the user device 130 or the database 140 via a cable or a fiber. In some embodiments, the intelligent robot 110 may update or download a map stored in the database 140 based on the communication between the intelligent robot 110 and the database 140. For example, the intelligent robot 110 may acquire information on a route, and analyze the information to construct a map. In some embodiments, a whole map may be stored in the database 140. In some embodiments, the map constructed by the intelligent robot 110 may include information corresponding to a portion of the whole map. In some embodiments, the corresponding portion of the whole map may be updated based on the constructed map. When the intelligent robot 110 determines its destination and a current location, the whole map stored in the database 140 may be accessed by the intelligent robot 110. The portion of the whole map, including the destination and the current location of the intelligent robot 110, may be selected by the intelligent robot 110 to plan the route. In some embodiments, the intelligent robot 110 may plan the route based on the selected map, the destination, and the current location of the intelligent robot 110. In some embodiments, the intelligent robot 110 may use a map from the user device 130. For example, the user device 130 may download a map from the Internet. The user device 130 may direct a movement of the intelligent robot 110 based on the map downloaded from the Internet. As another example, the user device 130 may download a latest map from the database 140. Once the destination and the current location of the intelligent robot 110 are determined, the user device 130 may send the map obtained from the database 140 to the intelligent robot 110. In some embodiments, the user device 130 may be a part of the intelligent robot 110. In some embodiments, if the map constructed by the intelligent robot 110 includes its destination and the current location, the intelligent robot 110 may plan the route based on the constructed map.

The network 120 may be a single network or a combination of different networks. For example, the network 120 may be a local area network (LAN), a wide area network (WAN), a public network, a private network, a wireless local area network (WLAN), a virtual network, a metropolitan area network (MAN), a public switched telephone network (PSTN), or any combination thereof. For example, the intelligent robot 110 may communicate with the user device 130 and the database 140 via Bluetooth. The network 120 may also include various network access points. For example, a wired or wireless access point, such as a base station or an Internet switching point, may be included in the network 120. The user may send a control operation from the user device 130 to the intelligent robot 110, and receive a result via the network 120. The intelligent robot 110 may access the information stored in the database 140 directly or via the network 120.

The user device 130 connectable to the network 120 may be a mobile device 130-1, a tablet computer 130-2, a laptop computer 130-3, a built-in device 130-4, or the like, or any combination thereof. In some embodiments, the mobile device 130-1 may include a wearable device, an intelligent mobile device, a virtual reality device, an augmented reality device, or the like, or any combination thereof. In some embodiments, the user may control the intelligent robot 110 via a wearable device. The wearable device may include an intelligent bracelet, an intelligent footwear, intelligent glasses, an intelligent helmet, an intelligent watch, an intelligent wear, an intelligent backpack, an intelligent accessory, or the like, or any combination thereof. In some embodiments, the intelligent mobile device may include an intelligent phone, a personal digital assistant (PDA), a gaming device, a navigation device, a point of sale (POS) device, or the like, or any combination thereof. In some embodiments, the virtual reality device and/or the augmented reality device may include a virtual reality helmet, virtual reality glasses, virtual reality eyewear, an augmented reality helmet, augmented reality glasses, augmented reality eyewear, or the like, or any combination thereof. For example, the virtual reality device and/or the augmented reality device may include Google Glass, Oculus Rift, HoloLens, Gear VR, or the like. In some embodiments, the built-in device 130-4 may include a laptop computer, a car TV, or the like. In some embodiments, the user device 130 may be a device having a positioning technique for determining a location of the user and/or the user device 130 regarding the user. For example, the intelligent robot 110 may determine the route based on the map, the destination, and the current location of the intelligent robot 110. The location of the intelligent robot 110 may be obtained by the user device 130. In some embodiments, the user device 130 may be a device having image capture capabilities. For example, the map stored in the database 140 may be updated based on information acquired by an imaging sensor (e.g., a camera). In some embodiments, the user device 130 may be a part of the intelligent robot 110. For example, a smartphone with a camera, a gyroscope, and an accelerometer may be held by the tripod head of the intelligent robot 110. The user device 130 may be designated as a sensor to detect information. As another example, a processor 210 and a storage 220 may be the portions of the smartphone. In some embodiments, the user device 130 may also be designated as a communication interface for the user of the intelligent robot 110. For example, the user may touch a screen of the user device 130 to select the control operation of the intelligent robot 110.

The database 140 may store the whole map. In some embodiments, a plurality of intelligent robots may be wirelessly connected to the database 140. Each intelligent robot connected to the database 140 may construct the map based on the information acquired by the sensors. In some embodiments, the map constructed by the intelligent robot may be a portion of the whole map. During a update process, the constructed map may replace a corresponding region in the whole map. When the route needs to be planned from the location of the intelligent robot 110 to the destination, each intelligent robot may download the map from the database 140. In some embodiments, the map downloaded from the database 140 may be a portion of the whole map that at least includes the location and the destination of the intelligent robot 110. The database 140 may also store historical information regarding the user connected to the intelligent robot 110. The historical information may include, for example, a previous operation of the user or information regarding how to operate the intelligent robot 110. As shown in FIG. 1, the database 140 may be accessed by the intelligent robot 110 and the user device 130.

It should be noted that the intelligent robot control system 100 is merely intended to describe one example of a particular embodiment of the system and is not intended to limit the scope of the disclosure.

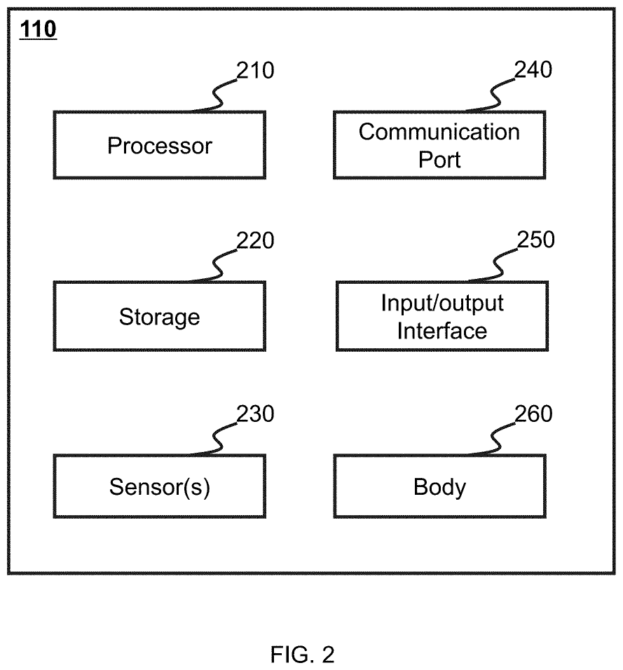

FIG. 2 is a schematic diagram illustrating an intelligent robot 110 in the intelligent robot control system illustrated in FIG. 1 according to some embodiments of the present disclosure. The intelligent robot 110 may include a processor 210, a storage 220, a sensor(s) 230, a communication port 240, an input/output interface 250, and a body 260. The sensor(s) 230 may obtain information. In some embodiments, the information may include image data, gyroscope data, accelerometer data, position data, and distance data. The processor 210 may process the information to generate one or more results. In some embodiments, the one or more results may include displacement information and depth information (e.g., the displacement of a camera between two adjacent frames and the depth of an object in two adjacent frames). In some embodiments, the processor 210 may construct a map based on the one or more results. The processor 210 may also transmit the map to the database 140 for further updating. In some embodiments, the processor 210 may include one or more processors (e.g., a single core processor or a multi-core processor). For example, the processor 210 may include a central processing unit (CPU), an application specific integrated circuit (ASIC), a dedicated instruction set processor (ASIP), a graphics processing unit (GPU), a physical processing unit (PPU), a digital signal processor (DSP), a field programmable gate array (FPGA), a programmable logic device (PLD), a controller, a microcontroller unit, a reduced instruction set computer, a microprocessor, or the like, or any combination thereof.

The storage 220 may store instructions for the processor 210. When executing the instructions, the processor 210 may perform one or more functions or operations described in the present disclosure. For example, the storage 220 may store instructions that may be executed by the processor 210 to process the information obtained by the sensor(s) 230. In some embodiments, the processor 220 may automatically store the information obtained by the sensor(s) 230. The storage 220 may also store the one or more results generated by the processor 210 (e.g., the displacement information and/or the depth information used to construct the map). For example, the processor 210 may generate the one or more results and store them in the storage 220. The processor 210 may read the one or more results from the storage 220, and construct the map. In some embodiments, the storage 220 may store the map constructed by the processor 210. In some embodiments, the storage 220 may store the map from the database 140 or the user device 130. For example, the storage 220 may store the map constructed by the processor 210. The constructed map may be transmitted to the database 140 to update the corresponding portion of the whole map. As another example, the storage 220 may temporarily store the map downloaded by the processor 210 from the database 140 or the user device 130. In some embodiments, the storage 220 may include a mass storage, a removable storage, a volatile read/write storage, a read-only storage (ROM), or the like, or any combination thereof. The exemplary mass storage may include a disk, an optical disk, s solid-state drive, or the like. The exemplary removable storage may include a flash drive, a floppy disk, an optical disk, a memory card, a zip disk, a magnetic tape, or the like. The exemplary volatile read/write storage may include a random-access storage (RAM). The exemplary RAM may include a dynamic RAM (DRAM), a dual date rate synchronous dynamic RAM (DDR SDRAM), a static RAM (SRAM), a thyristor RAM (T-RAM), a zero capacitor RAM (Z-RAM), or the like. The exemplary ROM may include a mask ROM (MROM), a programmable ROM (PROM), an erasable programmable ROM (EPROM), an electrically erasable programmable ROM (EEPROM), a compact disk ROM (CD-ROM) digital multifunction disk ROM.

The sensor(s) 230 may obtain image data of an object or an obstacle, gyroscope data, accelerometer data, position data, distance data, and other data that may be used by the intelligent robot 110 to perform the various functions described in this disclosure. For example, the sensor(s) 230 may include one or more night-vision cameras for obtaining the image data in low-light surroundings. In some embodiments, the data and/or the information obtained by the sensor(s) 230 may be stored in the storage 220 and may be processed by the processor 210. In some embodiments, the one or more sensor(s) 230 may be disposed in the body 260. For example, one or more imaging sensors may be disposed in the tripod head of the body 260. One or more navigation sensors, a gyroscope, and an accelerometer may be disposed in the tripod head and a motion assembly. In some embodiments, the sensor(s) 230 may automatically sense the surroundings and detect a location under the control of the processor 210. For example, the sensor(s) 230 may be used to dynamically sense or detect the location of the object, the obstacle, and so on.

The communication port 240 may be a communication port for communicating in the intelligent robot 110. That is, the communication port 240 may exchange information between components of the intelligent robot 110. In some embodiments, the communication port 240 may send signals/data/signals from the processor 210 to an internal component of the intelligent robot 110, and receive signals from the internal component of the intelligent robot 110. For example, the processor 210 may receive information from the sensor disposed in the body 260. As another example, the processor 210 may send a control operation to the body 260 via the communication port 240. The send-receive process may be implemented via the communication port 240. The communication port 240 may receive various wireless signals in accordance with a wireless communication specification. In some embodiments, the communication port 240 may be provided as a communication module for wireless local area communications, such as Wi-Fi, Bluetooth, infrared (IR), ultra-wideband (UWB), ZigBee, and so on, or as a mobile communication module, such as 3G, 4G, or Long Term Evolution (LTE), or as a communication method for a wired communication. In some embodiments, the communication port 240 may include but not limited to an element for sending to/receiving signals from an internal device, and may be used as an interface for interactive communications. For example, the communication port 240 may establish the communication between the processor 210 and other components of the intelligent robot 110 via a circuit of an application program interface (API). In some embodiments, the user device 130 may be a portion of the intelligent robot 110. In some embodiments, the communication between the processor 210 and the user device 130 may be performed by the communication port 240.

The input/output interface 250 may be an interface for communications between the intelligent robot 110 and other external devices, such as the database 140. In some embodiments, the input/output interface 250 may control data transmissions with the intelligent robot 110. For example, the latest map may be transmitted from the database 140 to the intelligent robot 110. As another example, the constructed map, based on the information obtained by the sensor(s) 230, may be transmitted from the database 140 to the intelligent robot 110. The input/output interface 250 may also include various components, such as a wireless communication module (not shown) for a wireless communication or a tuner (not shown) for adjusting broadcast signals, which may depend on a type of the intelligent robot 110 and the components used for receiving signals/data from external components. The input/output interface 250 may be used as the communication module for the wireless local area communications, such as the Wi-Fi, the Bluetooth, the infrared (IR), the ultra-wideband (UWB), the ZigBee, and so on, or as the mobile communication module, such as the 3G, the 4G or the Long Term Evolution (LTE), or as an input/output interface for the wired communication. In some embodiments, the input/output interface 250 may be provided as the communication module for a wired communication, such as an optical fiber or a Universal Serial Bus (USB). For example, the intelligent robot 110 may exchange data with the database 140 of a computer via a USB interface.

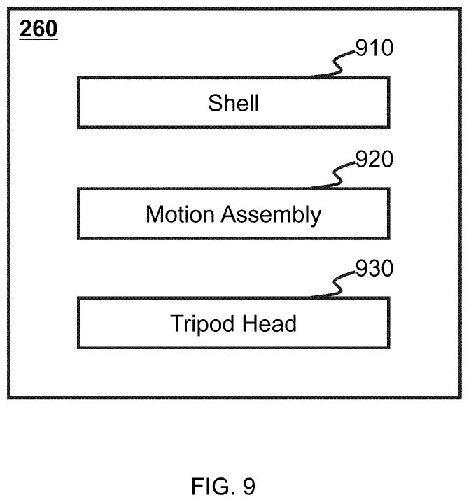

The body 260 may be a body for holding the processor 210, the storage 220, the sensor(s) 230, the communication port 240, and the input/output interface 250. The body 260 may execute instructions from the processor 210 to move and rotate the sensor(s) 230 to obtain or detect the information of a region. In some embodiments, the body 260 may include the motion assembly and the tripod head, as described elsewhere in the present disclosure (e.g., FIG. 9 and descriptions thereof). In some embodiments, sensor(s) may be disposed in the motion assembly and the tripod head, respectively.

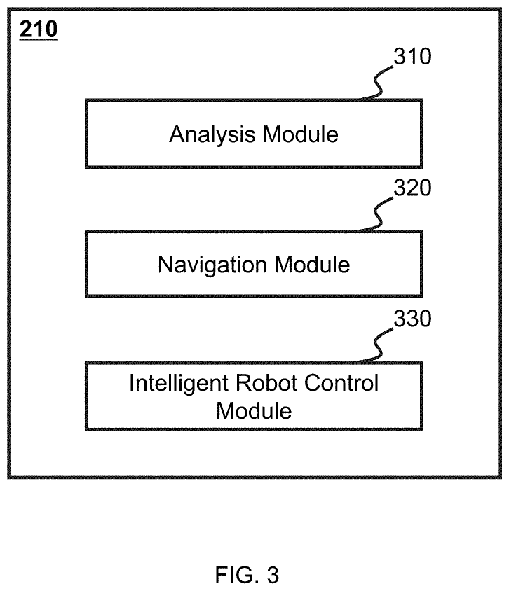

FIG. 3 is a schematic block diagram illustrating a processor 210 according to some embodiments of the present disclosure. As shown in FIG. 3, the processor 210 may include an analysis module 310, a navigation module 320, and an intelligent robot control module 330.

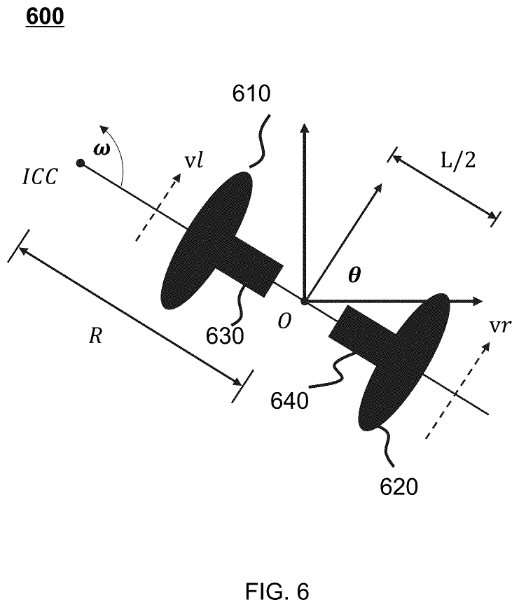

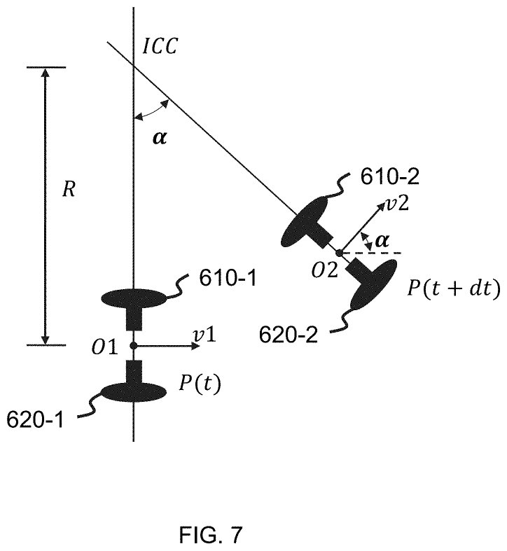

The analysis module 310 may analyze the information obtained from the sensor(s) 230, and generate one or more results. The analysis module 310 may construct the map based on the one or more results. In some embodiments, the constructed map may be sent to the database 140. In some embodiments, the analysis module 310 may receive the latest map from the database 140 and send it to the navigation module 320. The navigation module 320 may plan the route from the location of the intelligent robot 110 to the destination. In some embodiments, the whole map may be stored in the database 140. The constructed map may correspond to a portion of the whole map. A update process may include replacing the corresponding portion of the whole map with the constructed map. In some embodiments, the constructed map may be latest, and may include the location and the destination of the intelligent robot 110. The analysis module 310 may not receive the map from the database 140. The constructed map may be transmitted to the navigation module 320 to plan the route. The intelligent robot control module 330 may generate control parameters of the intelligent robot 110 based on the route planned by the navigation module 320. In some embodiments, the control parameters may be temporarily stored in the storage 220. In some embodiments, the control parameters may be sent to the body 260 to control the movement of the intelligent robot 110. More descriptions about the control parameters may be found elsewhere in the present disclosure (e.g., FIG. 6, FIG. 7, and the descriptions thereof).

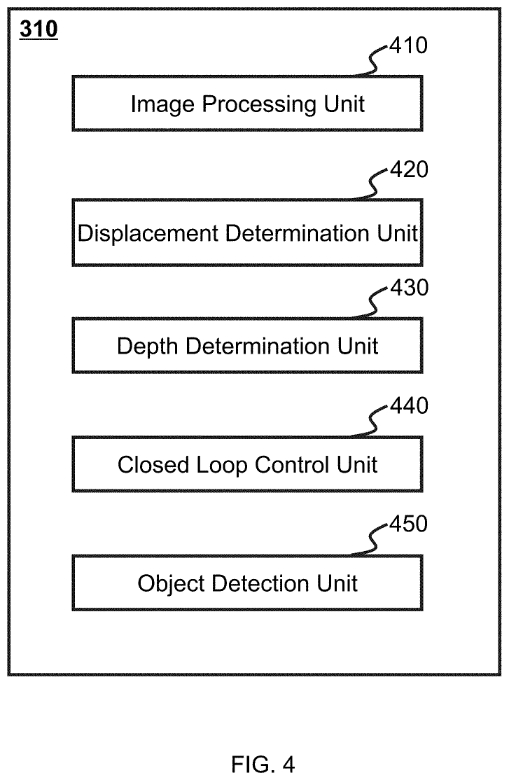

FIG. 4 is a schematic diagram illustrating an analysis module in the processor 210 illustrated in FIG. 3 according to some embodiments of the present disclosure. In some embodiments, the analysis module 310 may include an image processing unit 410, a displacement determination unit 420, a depth determination unit 430, a closed loop control unit 440, and an object detection unit 450.

The image processing unit 410 may process image data to perform one or more functions of the intelligent robot 110. The image data may include, for example, one or more images (e.g., a still image, a video frame, etc.), an initial depth of each pixel point in each frame, an initial displacement, and/or other data regarding the one or more images. In some embodiments, the displacement may include a displacement of a wheel(s) and a displacement of the camera relative to the wheel(s) between the two time points at which two adjacent frames are taken. The image data may be provided by various devices capable of providing the image data, such as the sensor(s) 230 (e.g., one or more imaging sensors). In some embodiments, the image data may include data regarding a plurality of images. The image may include a video frame sequence (also referred to as a “frame”). Each frame may be a frame, a numeric field, and so on.

In some embodiments, the image processing unit 410 may process the image data to generate motion information of the intelligent robot 110. For example, the image processing unit 410 may process two frames (e.g., a first frame and a second frame) to determine a difference between the two frames. The image processing unit 410 may then generate the motion information of the intelligent robot 110 based on the difference between the frames. In some embodiments, the first frame and the second frame may be adjacent frames (e.g., a current frame and a previous frame, a current frame and a subsequent frame, etc.). In some embodiments, the first frame and the second frame may also be non-adjacent frames. Specifically, for example, the image processing unit 410 may determine one or more corresponding pixel points between the first frame and the second frame and one or more regions including the one or more corresponding pixel points (also referred to as “overlapping regions”). In response to a determination that the first pixel point(s) and the second pixel point(s) correspond to a same object, the image processing unit 410 may determine the first pixel point(s) in the first frame as the corresponding pixel point(s) of the second pixel point(s) in the second frame. The first pixel point(s) in the second frame and its corresponding pixel point(s) (e.g., the second pixel point(s)) may correspond to the same location of the object. In some embodiments, the image processing unit 410 may identify one or more pixel points in the first frame that do not have corresponding pixel points in the second frame. The image processing unit 410 may further identify one or more regions (also referred to as “non-overlapping regions”) including the identified pixel points. The non-overlapping regions may correspond to the motion of the sensor(s) 230. In some embodiments, the pixel points of the non-overlapping regions in the first frame having no corresponding pixel points in the second frame may be omitted in a further processing (e.g., the processing operations of the displacement determination unit 420 and/or the depth determination unit 430).

In some embodiments, the image processing unit 410 may determine an intensity of the pixel point in the first frame and the corresponding pixel point in the second frame. In some embodiments, the intensity of the pixel point in the first frame and the corresponding pixel point in the second frame may be designated as a criterion for determining the difference between the first frame and the second frame. For example, the intensity of RGB components may be selected as the criterion for determining the difference between the first frame and the second frame. The pixel points, the corresponding pixel points, and the intensity of the RGB components may be sent to the displacement determination unit 420 and/or the depth determination unit 430 for determining the displacement and the depth of the second frame. In some embodiments, the depth may represent a spatial depth of the object in the two frames. In some embodiments, the displacement information may include a set of displacements regarding a set of frames. In some embodiments, the depth information may include depths of the set of frames. The frames, the displacement information, and the depth information may be used to construct the map.

The displacement determination unit 420 may determine the displacement information based on the data provided by the image processing unit 410 and/or any other data. The displacement information may include one or more displacements that represent the motion information of the sensor(s) 230 (e.g., an imaging sensor for capturing a plurality of frames) configured to acquire the image data. For example, the displacement determination unit 420 may obtain the data regarding the corresponding pixel points in the two frames (e.g., the first frame and the second frame). The data may include one or more values of the pixel points, such as a gray value, an intensity value, and so on. The displacement determination unit 420 may determine the values regarding the pixel points according to any suitable color model (e.g., a RGB model, a hue, saturation, and brightness (HSV) model, etc.). In some embodiments, the displacement determination unit 420 may determine the difference between the corresponding pixel points in the two frames. For example, the image processing unit 410 may identify the first pixel points in the first frame and its corresponding pixel points (e.g., the second pixel points) in the second frame. The image processing unit 410 may determine the second pixel points according to coordinate transformation of the first pixel points. The first pixel points and the second pixel points may correspond to the same object. The displacement determination unit 420 may also determine the difference between the values of the first pixel points and the second pixel points. In some embodiments, the displacement may be determined by minimizing a sum of the differences between the corresponding pixel points in the first frame and the second frame.

In some embodiments, the displacement determination unit 420 may determine an initial displacement ξ_{ji,1} representing estimated value of the displacement from an origin. For example, the initial displacement ξ_{ji,1} may be determined according to Equation (1) as follows:

$${ξ}_{ji , 1} = {argmin}_{{ξ}_{ji}} {\int }_{Ω} {\vert {I}_{i} ( x ) - {I}_{j} ( ω ( x , {D}_{i} ( x ) , {ξ}_{j i} ) ) \vert}_{δ} dx , \qquad ( 1 )$$

where, x denotes coordinates of a pixel point in the first frame; ω(x,Dᵢ(x),ξⱼᵢ) denotes coordinates of a corresponding pixel point in the second frame, ω(x,Dᵢ(x),ξⱼᵢ) and Iᵢ(x) may be at the same position relative to an object, ω(x,Dᵢ(x),ξⱼᵢ) denotes a transformed pixel point of the x when a camera moves a certain displacement ξⱼᵢ. Ω denotes a set of a pixel point pair. Each pixel point pair may include a pixel point in the first frame and the corresponding pixel point in the second frame. Iᵢ(x) denotes a RGB intensity of the pixel point whose coordinates are x; Iⱼ(ω(x,Dᵢ(x),ξⱼᵢ)) denotes the RGB intensity of the pixel point ω(x,Dᵢ(x),ξⱼᵢ).

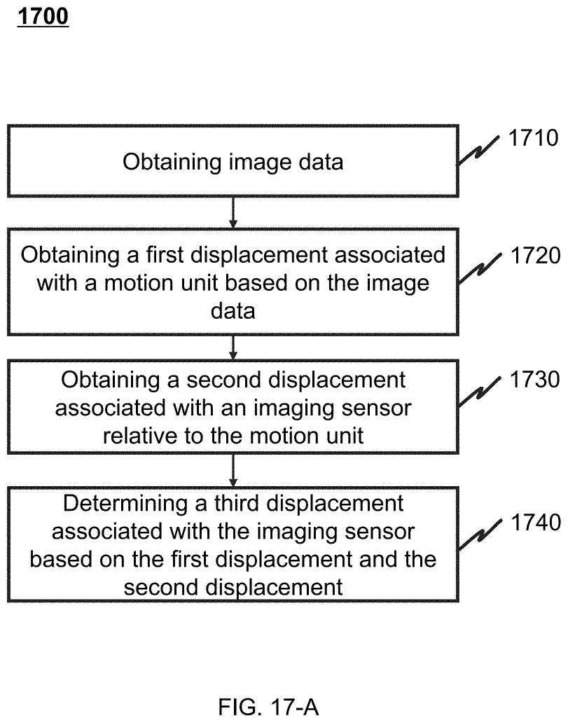

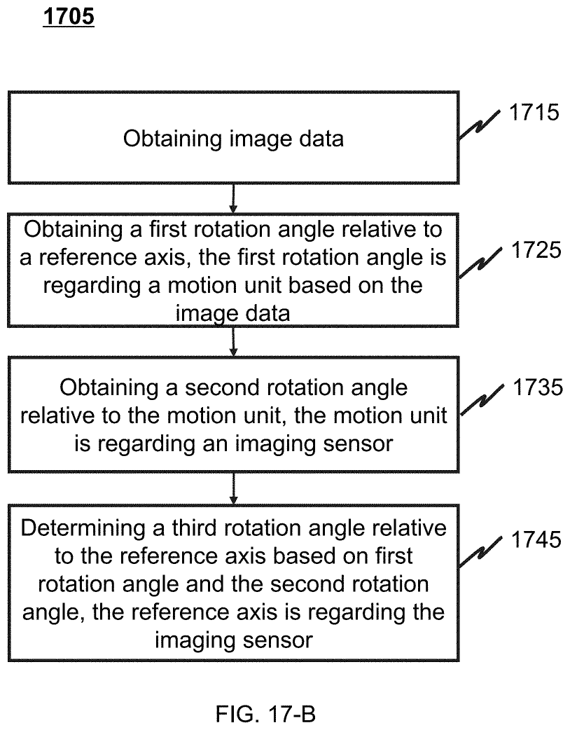

ω(x,Dᵢ(x),ξⱼᵢ) may denote the transformed coordinates of x when the camera moves a certain displacement ξⱼᵢ. In some embodiments, the displacement determination unit 420 may determine the corresponding pixel point ω(x,Dᵢ(x),ξⱼᵢ) based on the initial value of the displacement ξⱼᵢ and the initial depth Dᵢ(x). In some embodiments, the initial depth Dᵢ(x) may be a zero matrix. The initial value of the displacement ξⱼᵢ may be a variable. In order to obtain the initial displacement ξⱼᵢ, the displacement determination unit 420 may need the initial value ξⱼᵢ of the displacement as shown in an iterative Equation (1). In some embodiments, the initial value of the displacement ξⱼᵢ may be determined based on the displacement of the wheel ξⱼᵢ′ and the displacement of the camera relative to the wheel ξⱼᵢ′. More descriptions of the initial value ξⱼᵢ′ may be found elsewhere in the present disclosure (e.g., FIG. 17A, and the descriptions thereof). In some embodiments, the initial value of the displacement may be a sum of vector ξⱼᵢ′ and vector ξⱼᵢ″. The minimum difference between the two frames may be obtained according to the initial value and updated value of the displacement ξⱼᵢ.

In some embodiments, the depth determination unit 430 may determine updated depth D_{i,1}(x). The updated depth D_{i,1}(x) may be determined according to Equation (2) as follows:

$${D}_{i , 1} = {argmin}_{{D}_{i} ( x )} {\int }_{Ω} {\vert {I}_{i} ( x ) - {I}_{j} ( ω ( x , {D}_{i} ( x ) , {ξ}_{ji , 1} ) ) \vert}_{δ} dx , \qquad ( 2 )$$

where the depth Dᵢ(x) denotes a variable of a difference between two frames in the Equation (2). When the difference between the two frames is the smallest, the value D_{i,1}(x) may be determined as the updated depth. In some embodiments, the initial depth Dᵢ(x) may be a zero matrix.

The displacement determination unit 420 may also generate an updated displacement ξ_{ji,1u} based on the updated depth D_{i,1}(x). In some embodiments, the updated displacement ξ_{ji,1u} may be determined by replacing the initial depth Dᵢ(x) with the updated depth D_{i,1}(x) according to the Equation (1).

The closed loop control unit 440 may perform a closed loop detection. The closed loop control unit 440 may detect whether the intelligent robot 110 returns to a previously visited location and update displacement information based on the detection result. In some embodiments, in response to a determination that the intelligent robot 110 has returned to the previously visited location, the closed loop control unit 440 may adjust the updated displacement of the frame to reduce an error by using a g2o closed loop detection. The g2o closed loop detection may be a general optimization framework for reducing the nonlinear error. The adjusted updated displacement of the frame may be designated as the displacement information. In some embodiments, the intelligent robot 110 may include a depth sensor, such as a laser radar, and the depth may be obtained directly. The displacement may be determined according to the Equation (1). The closed loop control unit 440 may adjust the displacement to generate the adjusted displacement.

When the depth sensor detects the depth information, the displacement information may be a set of displacements determined according to the Equation (1). The displacement information may be adjusted by the closed loop control unit 440. When the depth information is a set of updated depths, the displacement information may be a set of displacements adjusted according to the Equation (1), the Equation (2), and by the closed loop control unit 440.

In some embodiments, the closed loop control unit 440 may generate the map according to the frames, the displacements, and the depth information.

The analysis module 310 may also include the object detection unit 450. The object detection unit 450 may detect obstacles, objects, and distances from the intelligent robot 110 to the obstacles and the objects. In some embodiments, the obstacles and the objects may be determined based on the data obtained by the sensor(s) 230. For example, the object detection unit 450 may detect the object based on distance data acquired by a sonar, an infrared distance sensor, an optical flow sensor, or a laser radar.

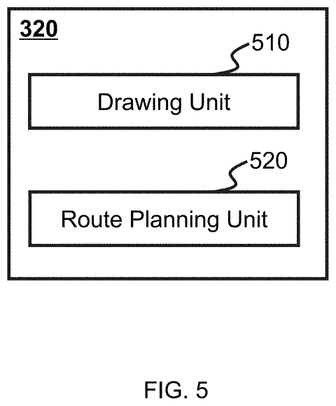

FIG. 5 is a schematic block diagram illustrating an exemplary navigation module 320 in the processor 210 according to some embodiments of the present disclosure. In some embodiments, the navigation module 320 may include a drawing unit 510 and a route planning unit 520. In some embodiments, the drawing unit 510 may obtain a map from the database 140. In some embodiments, the drawing unit 510 may process the map for a route planning. In some embodiments, the map may be a portion of a whole map in the database 140. For example, the map may include a destination and a current location of the intelligent robot 110, and the map may be used to plan a route. In some embodiments, the map obtained from the database 140 may be a three-dimensional map. In some embodiments, the drawing unit 510 may convert the three-dimensional map into a two-dimensional map according to a projection technique. The drawing unit 510 may divide an object in the three-dimensional map to pixel points, and project the pixel points to a horizontal surface to generate the two-dimensional map. When the drawing unit 510 determines the two-dimensional map, the route planning unit 520 may plan the route from the current location of the intelligent robot 110 to the destination based on the two-dimensional map.

The intelligent robot control module 330 may determine control parameters based on the route planned by the route planning unit 520 in the navigation module 320. In some embodiments, the intelligent robot control module 330 may divide the route to a group of segments. The intelligent robot control module 330 may obtain a set of nodes for the segments. In some embodiments, the node between two segments may be an end point of a previous segment and/or a starting point of a current segment. The control parameters of the segment may be determined based on the start point and/or the end point.

In some embodiments, during the movement of the intelligent robot 110 in a segment, if an end point of the intelligent robot 110 does not match a predetermined end point of the segment, the route planning unit 520 may plan a new route based on the mismatched end point (as a new start location of the intelligent robot 110) and the destination. In some embodiments, the intelligent robot control module 330 may divide the new route and generate one or more new segments. The intelligent robot control module 330 may then determine a set of control parameters for each new segment.

FIG. 6 and FIG. 7 are schematic diagrams illustrating exemplary motion controls of the intelligent robot 110. As shown in FIG. 6, a motion assembly may move around a point ICC at an angular velocity ω. The motion assembly may have two wheels. Left wheel 610 may move at a speed v₁ and right wheel 620 may move at a speed vᵣ. In some embodiments, a distance between the left wheel 610 and the right wheel 620 may be L. Either a distance between the left wheel 610 and a center point O or a distance between the right wheel 620 and the center point O may be L/2. A distance between the center point O and the point ICC may be R.

FIG. 7 is a schematic diagram illustrating an exemplary process for determining control parameters of the intelligent robot 110. As shown in FIG. 7, the motion assembly of the intelligent robot 110 may move from a point O₁ to a point O₂ within dt. An included angle between a line connecting the point O₁ and the point ICC and a line connecting the point O₂ and the point ICC may be α. If dt, L, R, and α are known, a speed of the left wheel vₗ and a speed of the right wheel vᵣ may be determined accordingly.

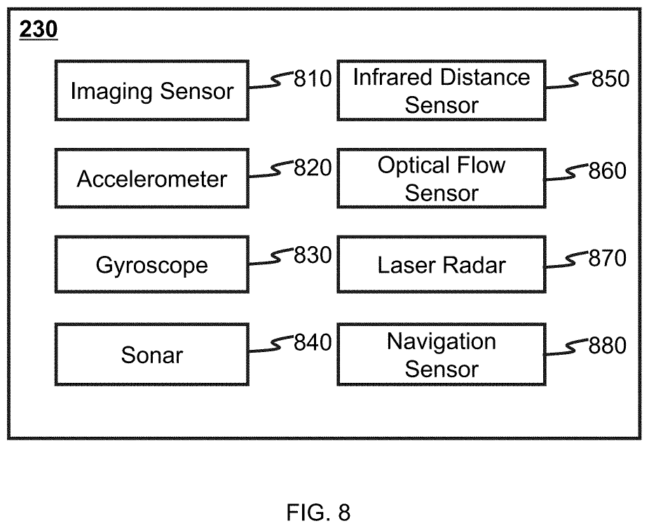

FIG. 8 is a schematic diagram illustrating a component of sensor(s) 230 according to some embodiments of the present disclosure. The sensor(s) 230 may include an imaging sensor 810, an accelerometer 820, a gyroscope 830, a sonar 840, an infrared distance sensor 850, an optical flow sensor 860, a laser radar 870, and a navigation sensor 880.

The imaging sensor 810 may acquire image data. In some embodiments, the analysis module 310 may construct a map based on the image data. In some embodiments, the image data may include a frame, an initial depth and an initial displacement of each pixel point of each frame. In some embodiments, the initial depth and the initial displacement may be used to determine a depth and a displacement of the pixel point. More descriptions about obtaining the depth and the displacement may be found elsewhere in the present disclosure (e.g., Equation (1) in FIG. 4, and the descriptions thereof). In some embodiments, the displacement may include a displacement of the wheel and/or a displacement of a camera relative to the wheel between the two time points at which two adjacent frames are taken.

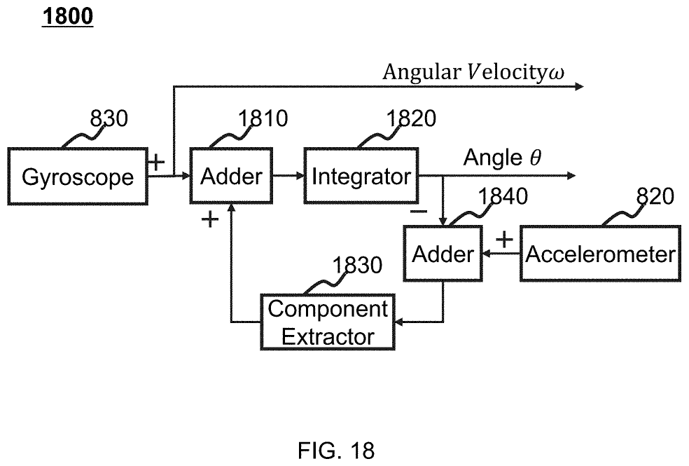

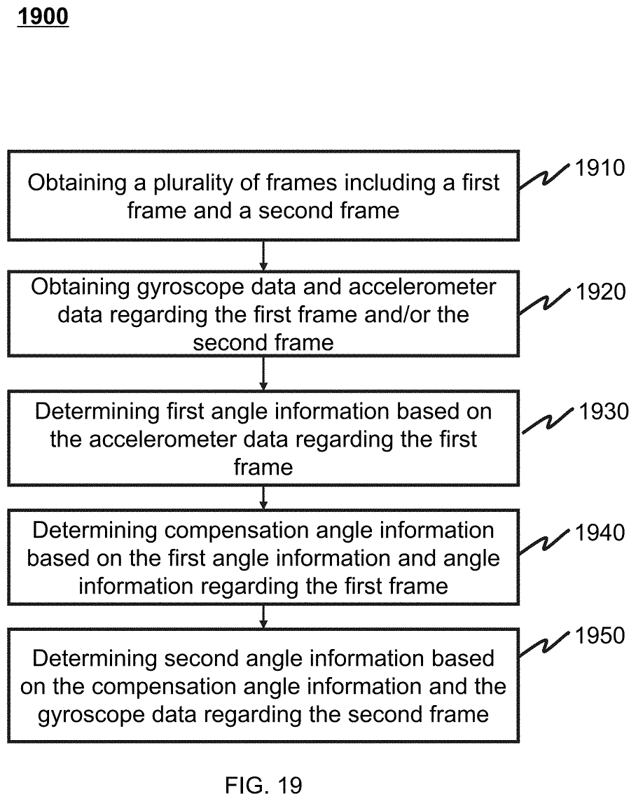

In order to keep balance between the motion assembly and the tripod head, the accelerometer 820 and the gyroscope 830 may be operated together. It is necessary to keep the balance aiming at obtaining stability information from the sensor(s) 230. In some embodiments, the accelerometer 820 and the gyroscope 830 may be operated together to control a pitch attitude within a certain threshold. In some embodiments, the accelerometer 820 and the gyroscope 830 may be held by the motion assembly and the tripod head, respectively. More descriptions about keeping balance may be found elsewhere in the present disclosure (e.g., FIG. 18 or FIG. 19, and the descriptions thereof).

The sonar 840, the infrared distance sensor 850, and the optical flow sensor 860 may be configured to determine the location of the intelligent robot 110. In some embodiments, the intelligent robot 110 may be positioned by one or a combination of the sonar 840, the infrared distance sensor 850, and the optical flow sensor 860.

The laser radar 870 may be configured to detect a depth of the object in a frame. The laser radar 870 may obtain the depth of each frame. In some embodiments, the analysis module 310 in the processor 210 may not determine the depth. The depth obtained by the laser radar 870 may be directly used to determine the displacement according to Equation (1) illustrated in FIG. 4. The closed loop control unit 440 may further adjust the determined displacement.

The sonar 840, the infrared distance sensor 850, and the optical flow sensor 860 may detect the distance between the intelligent robot 110 and the object or the obstacle. Thus, the intelligent robot 110 may be positioned. The navigation sensor 880 may position the intelligent robot roughly. In some embodiments, the navigation sensor 880 may position the intelligent robot 110 with any type of positioning system. The positioning system may include a Global Positioning System (GPS), a Beidou navigation or positioning system, and a Galileo positioning system.

In some embodiments, when the robot is embodied in the form of an intelligent wheelchair, the sensor group may also include a set of medical monitoring sensors. The medical monitoring sensor may monitor and record the user's physiological information. The medical monitoring sensor may be in contact with the user's body surface. When the medical monitoring sensor is connected to the user's body surface, the intelligent wheelchair may continuously monitor the user's physiological information in real time (or near real time), and transmit monitoring results to external devices (including but not limited to a storage device or a cloud server). For example, the intelligent wheelchair may continuously monitor the physiological information of the user within a random period, such as minutes, hours, days, or months. The intelligent wheelchair may also periodically monitor the user's physiological information.

In some embodiments, the intelligent wheelchair or a third party processor (e.g., the processor 210) may compare a current physiological information of the user with a predetermined safety threshold. If an abnormality occurs, the processor (e.g., the processor 210) may generate a warning signal. The processor (e.g., the processor 210) may send the warning signal to a smart device, such as a smartphone or a tablet computer. The smart device may generate an alarm, such as a sound, a light, a vibration, to notify the user or other person (e.g., a family member of the user, relatives or friends, etc.). The alarm may remind a medical staff or the user to pay attention to an abnormality regarding a physiological index.

In some embodiments, the intelligent wheelchair or the third party processor (e.g., the processor 210) may predict future physiological information based on the user's historical physiological information. The processor may compare the predicted physiological information with the predetermined safety threshold. If an abnormality occurs, the processor (e.g., the processor 210) may determine an early warning signal. The processor (e.g., the processor 210) may send the early warning signal to a smart device, such as a smartphone or a tablet computer. The smart device may generate an alarm, such as sound, light, vibration, to notify the user or other person (e.g., a family member of the user, relatives or friends, etc.). The alarm may remind a medical staff or the user to pay attention to an abnormality regarding a physiological index occurred in the future. A corresponding security protection may be prepared.

In some embodiments, the predetermined safety threshold may include at least one of a safe blood pressure value, a safe blood oxygen level value, a safe heart rate value, or a safety pulse rate value.

In some embodiments, the intelligent wheelchair may display the monitored data in real time or non-real time. In some embodiments, the monitored information may be transmitted to a relevant third party via a wireless or a wired communication, such as, a hospital, a care facility, or a related person, and displayed on the third party's display device. In some embodiments, the monitored data may be stored in a local or remote storage device. For example, the monitored data may be stored in the storage 220 of the intelligent wheelchair, or may be stored in a third party's storage device, such as a database of a hospital or a care facility.

In some embodiments, the medical monitoring sensor is configured to collect the user's physiological information. The medical monitoring sensor may be implemented according to a photoelectric sensing technique or by electrode sensing a technique. The medical monitoring sensor may obtain physiological information by sensing a temperature change, a humidity change, a pressure change, a photoelectric induction, a body surface potential change, a voltage change, a current change, or a magnetic field change. The medical monitoring sensor may obtain various forms of information, such as acoustics, optics, magnetism, and thermal. The type of information may include but not limited to electrocardiographic information, heart rate information, pulse rate information, blood pressure information, blood oxygenation information, respiratory information, invasive blood pressure information, non-invasive blood pressure information, cardiac output, body temperature information, blood gas information, and so on. For example, the obtained electrocardiographic information may include but not limited to waveforms, time intervals, peaks, troughs, amplitudes, and so on.

In some embodiments, the medical monitoring sensor may include various devices, for example, a blood pressure measuring device, an electrocardiogram (ECG) monitoring device, a blood measuring device, a pulse wave detector, a brain wave monitor, a heart rate detector, a blood oxygen detector, a blood oxygen detector, a respiratory detector, an invasive blood pressure detector, a non-invasive blood pressure detector, a cardiac output detector, a body temperature detector, a blood gas detector, etc. The blood pressure measuring device may include but not limited to a watch type of blood pressure meter, a wrist type of blood pressure meter, an upper arm type of blood pressure meter, and so on. The ECG monitoring device may include but not limited to a medical ECG monitoring system, an ECG monitor, and so on. The medical monitoring sensor may, via a local processor, such as the processor 210, process the monitored data. The medical monitoring sensor may be wirelessly connected to a remote monitoring system. The remote monitoring system may include a medical monitoring system, or a domestic portable monitoring device. The medical monitoring sensor may be a conventional ECG monitoring device, or a portable smart wearable device, such as a watch or a headphone having the monitoring function. The medical monitoring sensor may acquire the whole physiological information. The medical monitoring sensor may acquire the physiological information in a time interval, such as, a 4 seconds time window.

In some embodiments, the medical monitoring sensor may be integrated to the intelligent wheelchair system. In some embodiments, the medical monitoring sensor may be connected to an external device of the intelligent wheelchair system through an input/output (I/O) interface, such as a conventional monitoring device or a portable wearable monitoring device.

FIG. 9 is an exemplary block diagram of the body 260 illustrated in FIG. 2 according to some embodiments of the present disclosure. The body 260 may include a shell 910, a motion assembly 920, and a tripod head 930. The shell 910 refers to a shell of the body 260. The shell of the body 260 may be configured to protect the modules and the units integrated to the intelligent robot 110. The motion assembly 920 refers to motion operating component(s) in the intelligent robot 110. In some embodiments, the motion assembly 920 may move based on control parameters generated by the intelligent robot control module 330 in the processor 210. For example, for a segment of a route determined by the intelligent robot control module 330, the control parameters may be determined based on a start point and an end point of the segment of the route. The control parameters may be transmitted from the intelligent robot control module 330 to the motion assembly 920. The intelligent robot 110 may move from the start point to the end point. In some embodiments, the tripod head 930 may include at least one support device for holding the sensor described in FIG. 8. The tripod head 930 may support the imaging sensor 810, such as a camera, to obtain a frame. In some embodiments, the tripod head may support the imaging sensor 810, such as a camera, to acquire the frame. In some embodiments, the tripod head 930 may support the accelerometer 820 and the gyroscope 830. Due to the support of the tripod head, the balance of the sensors may be kept so as to obtain stability information. The tripod head 930 may support at least one of the sonar 840, the infrared distance sensor 850, and the optical flow sensor 860 which may detect a distance between the intelligent robot 110 and the object or the obstacle. The tripod head 930 may also support the laser radar 870 and other sensors which detect depth information or other information. In some embodiments, the navigation sensor 880 may be disposed on the tripod head 930. In some embodiments, the sensor supported by the tripod head may be integrated to a smartphone.

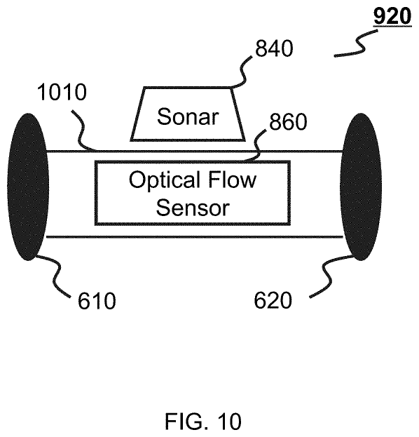

FIG. 10 is a schematic diagram illustrating the motion assembly 920 according to some embodiments of the present disclosure. The motion assembly 920 may include a motion unit and a carrier 1010. The motion unit may include two wheels. The two wheels may include the left wheel 610 and the right wheel 620. The carrier 1010 may be configured to hold the sonar 840 or the optical flow sensor 860 to detect an object or an obstacle. In some embodiments, the carrier 1010 may include the accelerometer 820 (not shown in FIG. 10) and the gyroscope 830 (not shown in FIG. 10) to keep balance of the motion assembly 920. In some embodiments, the carrier 1010 may include various sensors, such as the infrared distance sensor 850, to obtain information.

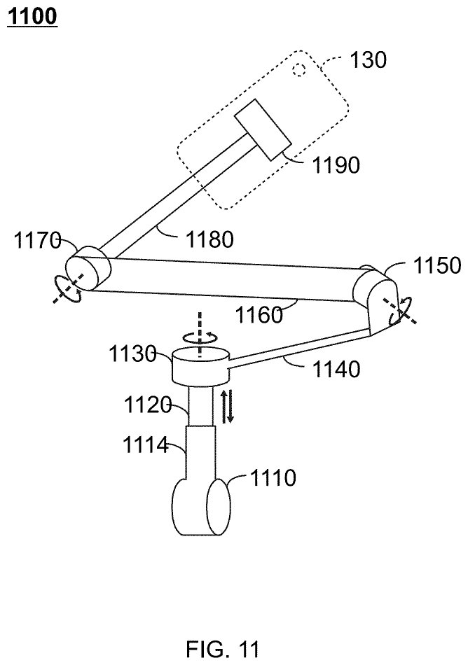

As shown in FIG. 9, the tripod head 930 may support the sensor(s) 230 to obtain the information to generate the map, plan the route, or generate the control parameters. FIG. 11 is an exemplary schematic diagram illustrating the tripod head 930 in the body 260 illustrated in FIG. 9 according to some embodiments of the present disclosure. In some embodiments, the tripod head 930 may include a first rotary shaft 1170 for controlling a rotation around a X axis, a second rotary shaft 1150 for controlling a rotation around a Y axis, and a third rotary shaft 1130 for controlling a rotation around a Z axis. The X axis may be a first axis in the horizontal plane, the Y axis may be a second axis in the horizontal plane, the Z axis may be a vertical axis perpendicular to the horizontal plane. In some embodiments, the tripod head 930 may include a connecting rod 1180 for connecting the rotary shaft 1170 and the sensor, a connecting rod 1160 for connecting the rotary shaft 1150 and the rotary shaft 1170, and a connecting rod 1140 for connecting the rotary shaft 1130 and the rotary shaft 1150. In some embodiments, the tripod head 930 may include a connector 1110, a connecting rod 1114, and a dynamic Z-buffer bar 1120. In some embodiments, the sensor may be integrated to the user device 130 (e.g., a smartphone). The user device 130 may include the sensor, such as the imaging sensor 810, the accelerometer 820, the gyroscope 830, and the navigation sensor 880. The tripod head 930 may also include a connection block 1190 to support the user device 130. During the operation of the tripod head 930, the sensor in the user device 130 may obtain information. In some embodiments, the sensor in the user device 130 may be controlled by adjusting an orientation of the tripod head 930 in order to obtain suitable information. In some embodiments, the orientation of the tripod head 930 may be adjusted by rotating the rotary shaft 1170, the rotary shaft 1150, and the rotary shaft 1130 around the X, Y, and Z axes.

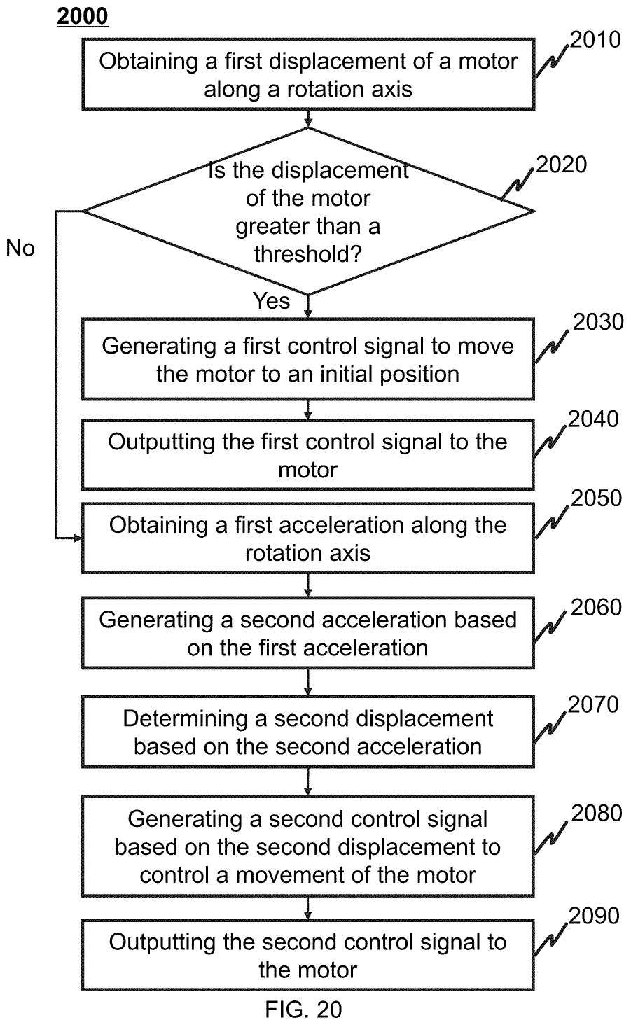

A traditional 3-axis tripod head may be used for aerial photography. The dynamic Z-buffer connecting rod 1120 may be used in the tripod head 930 to maintain the stability of the tripod head 930 during the motion. The dynamic Z-buffer connecting rod 1120 may maintain the stability of the tripod head 930 along the Z axis. In some embodiments, the dynamic Z-buffer connecting rod 1120 may be a telescopic rod that may expand and contract along the Z axis. An operating method of the dynamic Z-buffer connecting rod 1120 in the tripod head 930 is described in FIG. 20. The rotation and vertical movement of the rotary shafts 1130, 1150, and 1170 of the dynamic Z-buffer connecting rod 1120 may be controlled based on the control parameters generated by the intelligent robot control module 330.

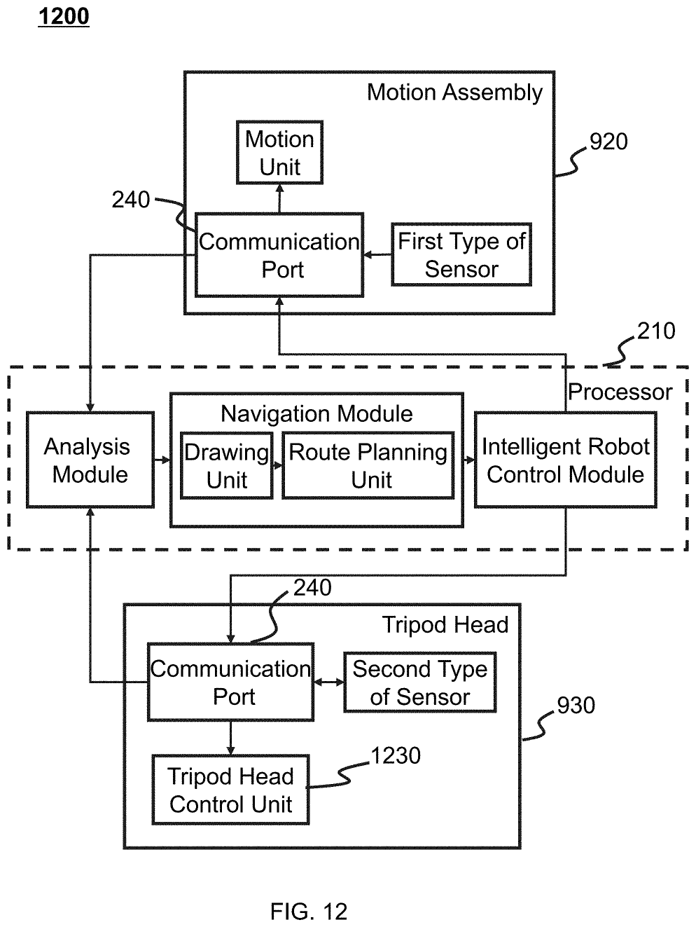

The intelligent robot 110 may have a plurality of modules and units. FIG. 12 illustrating a simplified system of the intelligent robot 110 according to some embodiments of the present disclosure. As shown in FIG. 12, the intelligent robot 110 may include the processor 210, the motion assembly 920, and the tripod head 930. In some embodiments, the processor 210 may include the analysis module 310, the navigation module 320, and the intelligent robot control module 330. The motion assembly 920 may include a motion unit 1210, a first type of sensor 1220, and the communication port 240. The tripod head 930 may include a tripod head control unit 1230, the communication port 240, and a second type of sensor 1240. In some embodiments, the processor 210 may send control parameters to control the motion unit 1210 in the motion assembly 920 and the tripod head control unit 1230 in the tripod head 930.

In some embodiments, the first type of sensor 1220 and the second type of sensor 1240 may obtain information. The analysis module 310 may process the obtained information and construct a map. In some embodiments, the constructed map may be sent to the database 140. In order to determine a route to the destination, the map for navigation may be required. The analysis module 310 may download a latest map from the database 140 and send the latest map to the navigation module 320. The navigation module 320 may process the latest map and determine the route from the location of the intelligent robot to the destination. In some embodiments, the analysis module 310 may not need to download the whole map. A portion of the whole map including the location of the intelligent robot and the destination may be sufficient for planning the route. In some embodiments, the map constructed by the analysis module 310 includes the location of the intelligent robot 110 and the destination. The map may be the latest map in the database. The map constructed by analysis module 310 may be sent to the navigation module 320 to plan the route. The navigation module 320 may include the drawing unit 510 and the route planning unit 520. In some embodiments, the drawing unit 510 may generate the two-dimensional map for planning route based on the latest map or the constructed map from the analysis module 310. The route planning unit 520 may plan the route. The route may be sent to the intelligent robot control module 330. The intelligent robot control module 330 may divide the route into one or more segments. The intelligent robot control module 330 may generate the control parameters for each segment of the route. Each segment may have the start point and the end point. The end point of the segment may be the start point of the next segment. In some embodiments, if the end point of the intelligent robot 110 in a segment does not match the predetermined end point of the segment, the later planning of the route may be impacted. It is necessary to re-plan the route based on the mismatched location (e.g., as a new location of the intelligent robot 110) and the destination. In some embodiments, once the mismatch issue is detected, the navigation module 320 may re-plan the route.

In some embodiments, if the first type of sensor 1220 in the motion assembly 920 and the second type of sensor 1240 in the tripod head 930 are not held stably, the information detected by the first type of sensor 1220 and the second type of sensor 1240 may not be used to construct the map accurately. The intelligent robot control module 330 may generate the control parameters to adjust the motion assembly 920 and the tripod head 930 in order to stabilize the first type of sensor 1220 and the second type sensor of 1240.

The sensors may be mounted on the motion assembly 920 and the tripod head 930. In some embodiments, the first type of sensor 1220 may include at least one of the accelerometer 820, the gyroscope 830, the sonar 840, the infrared distance sensor 850, the optical flow sensor 860, the laser radar 870, and the navigation sensor 880. In some embodiments, the second type of sensor 1240 may include at least one of the imaging sensor 810, the accelerometer 820, the gyroscope 830, the sonar 840, the infrared distance sensor 850, the optical flow sensor 860, the laser radar 870, and the navigation sensor 880.

As shown in FIG. 12, the processor 210 may establish a communication between the motion assembly and the tripod head 930 via the communication port 240. In some embodiments, the communication port 240 may exist in various forms. For example, the communication port 240 may be a wired or wireless transceiver. In some embodiments, the communication port 240 may exist in the form of an interface for interactive communication. For example, the communication port 240 may be configured to establish the communication between the processor 210 and other components of the intelligent robot 110 via a circuit of the application program interface (API). In some embodiments, the API may include a set of subroutine definitions, protocols, and tools for constructing software and application programs. In some embodiments, the API may make the development of the program simpler by providing the components. The components may be assembled together. In some embodiments, The API protocol may be used to design a circuit for a wireless communication. For example, the wireless circuit may include a Wi-Fi, a Bluetooth, an infrared (IR), a ultra-wideband (UWB), a wireless personal area network (ZigBee), and so on. The wireless circuit may also include a mobile communication module, such as 3G, 4G, and Long Term Evolution (LTE). The API may be configured to separate a bottom hardware (e.g., the motion assembly 920 or the tripod head) and a control hardware (e.g., the processing module 210). In some embodiments, the processing module 210 (e.g., a portion of the smartphone) may control the motion of the wheels in the motion assembly 920 and the pose of the imaging sensor (e.g., the camera) in the tripod head 930 by invoking the API in the communication port 240. In some embodiments, the first type of sensor 1220 in the motion assembly 920 may send information (e.g., location data) to the smartphone. In some embodiments, the second type of sensor 1240 in the tripod head 930 may send information (e.g., a pose of the camera) to the smartphone.

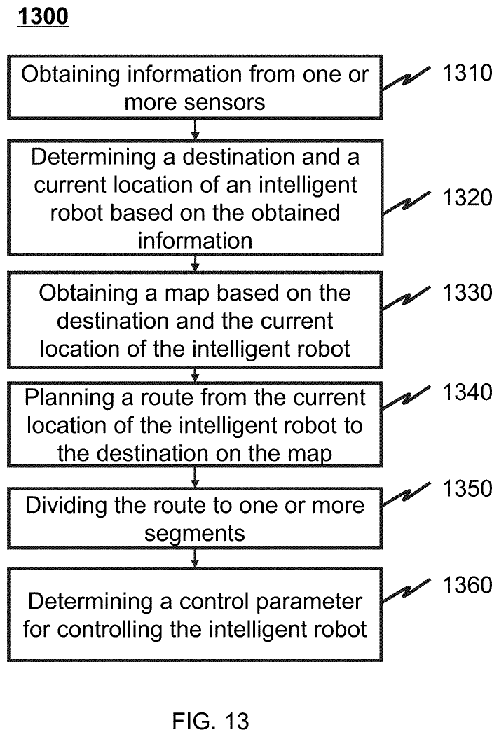

FIG. 13 is a flowchart illustrating an exemplary process for determining control parameters for controlling an intelligent robot according to some embodiments of the present disclosure. Process 1300 disclosed in FIG. 13 may be implemented by the processor 210 in the intelligent robot 110 by invoking and/or executing instructions stored in the storage 220.

In 1310, the processor 210 may obtain information from the sensor(s) 230. As described in connection with FIG. 3 and FIG. 12, the analysis module 310 in the processor 210 may receive the information from the motion assembly 920 and the tripod head 930 via the API communication port 240. In some embodiments, the movement of the intelligent robot 110 may be controlled by analyzing the information. In some embodiments, the stability of the motion assembly 920 and the tripod head 930 in the intelligent robot 110 may be maintained by analyzing the information.

In 1320, the processor 210 may determine a destination and a current location of the intelligent robot 110 based on the obtained information. For example, the analysis module 310 in the processor 210 may receive location data from the sensor(s) 230. The sensor may include but not limited to a sonar, an infrared distance sensor, an optical flow sensor, a laser radar, a navigation sensor, and so on. In some embodiments, a user may input the destination via the input/output (I/O) interface 250. For example, the user may input the destination for the intelligent robot 110. The processor 210 may plan a route for the intelligent robot 110 based on the user input destination. In some embodiments, the processor 210 may determine the current location of the intelligent robot 110 based on the obtained information. In some embodiments, the processor 210 may determine the current location of the intelligent robot 110 based on the information obtained from the sensor(s) 230. For example, the processor 210 may determine a rough location of the intelligent robot based on the information obtained by the navigation sensor 880 in the positioning system (e.g., GPS). As another example, the processor 210 may determine a precise location of the intelligent robot 110 based on the information obtained by at least one of the sonar 840, the infrared distance sensor 850, and the optical flow sensor 860.

In 1330, the processor 210 may obtain a map based on the destination and the current location of the intelligent robot 110. The map may be used to plan a route. In some embodiments, a whole map including a large number of points representing a city may be stored in the database 140. After determining the destination and the current location of the intelligent robot 110, the map including the destination and the current location of the intelligent robot 110 may be needed to plan the route from the current location to the destination. In some embodiments, the map including the destination and the current location of the intelligent robot 110 may be a part of the whole map. In some embodiments, the analysis module 310 in the processor 210 may obtain a suitable part of the whole map from the database 140 based on the destination and the current location of the intelligent robot 110. In some embodiments, the analysis module 310 may construct the map based on the information obtained from the sensor(s) 230. The constructed map may be sent to the database 140 to update the whole map. In some embodiments, the constructed map may include the destination and the current location of the intelligent robot 110. The navigation module 320 may use the constructed map to plan the route.

In 1340, the route from the current location of the intelligent robot 110 to the destination may be planned based on the map. The route planning may be performed by the navigation module 320. In some embodiments, the navigation module 320 may convert the obtained map into a two-dimensional map via the drawing unit 510. The route planning unit 520 may then determine the route from the current location of the intelligent robot 110 to the destination based on the two-dimensional map.

In 1350, the intelligent robot control module 330 may divide the planned route to one or more segments. Whether the route segmentation is performed or not may depend on a threshold. For example, if the planned route is less than the threshold, the route segmentation may not be performed. In some embodiments, the route segmentation may be performed by the intelligent robot control module 330 in response to the instructions stored in the storage module 220.

In 1360, the intelligent robot control module 330 may determine control parameters for controlling the intelligent robot based on the segmented route. In some embodiments, each segment may have a start point and an end point. In some embodiments, the intelligent robot control module 330 may determine the control parameters of the intelligent robot on the segment based on the start point and the end point of the segment. More descriptions of the determination of the control parameters between two points may refer back to the descriptions in FIG. 6 and FIG. 7. In some embodiments, the control parameters may be adjusted over time. For example, when the intelligent robot 110 passes through two points (e.g., from a first point to a second point) on a straight line on the segment, the intelligent robot 110 may use corresponding motion speeds in different time periods. In some embodiments, the control parameters may be used to keep the intelligent robot stable during the movement along the planned route. For example, due to the stability of the motion assembly 920 and the tripod head 930, the sensing information may be obtained accurately. As another example, in a case that the route is not flat, the control parameters may be used to stabilize the tripod head 930 in a direction perpendicular to the ground.

In some embodiments, when the intelligent robot passes the segment based on the predetermined control parameters, the intelligent robot 110 may stop at a location that does not match the predetermined end point of the segment determined by the intelligent robot control module 330. The navigation module 320 may re-plan a new route based on the mismatched location of the intelligent robot and the destination. The intelligent robot control module 330 may further divide the new planned route to one or more segments. The intelligent robot control module 330 may determine corresponding control parameters of the intelligent robot for the one or more segments. In some embodiments, when the intelligent robot 110 passes each segment, a matching error regarding the location may be estimated by comparing the actual location of the intelligent robot and the predetermined end point of the segment.

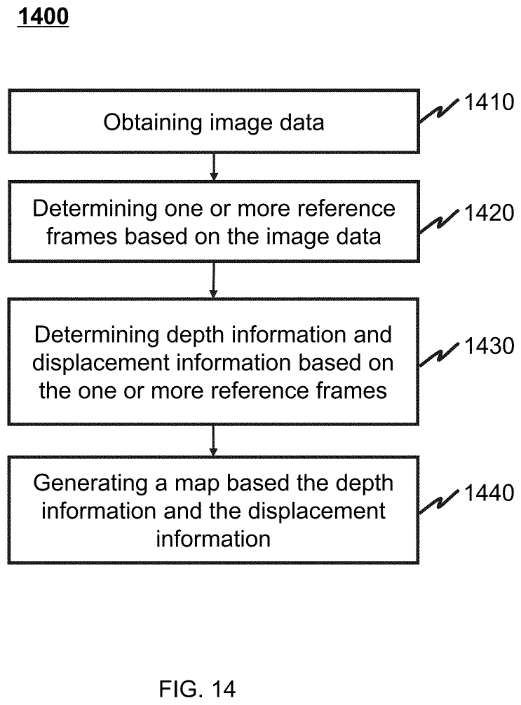

FIG. 14 is a flowchart illustrating an exemplary process for constructing a map according to some embodiments of the present disclosure. Process 1400 for constructing the map may be performed by the analysis module 310 based on the information obtained by the sensor(s) 230.

In 1410, the analysis module 310 may obtain image data from the imaging sensor 810. In some embodiments, the image data may include a large number of frames, an initial depth and/or a displacement of each pixel point in each frame. The displacement may include a displacement of the wheel and a displacement of the camera relative to the wheel. In some embodiments, the initial depth may be set as a zero matrix. In some embodiments, the sensor(s) 230 may include the laser radar or the camera with a depth detection function, and the sensor(s) 230 may obtain depth information.

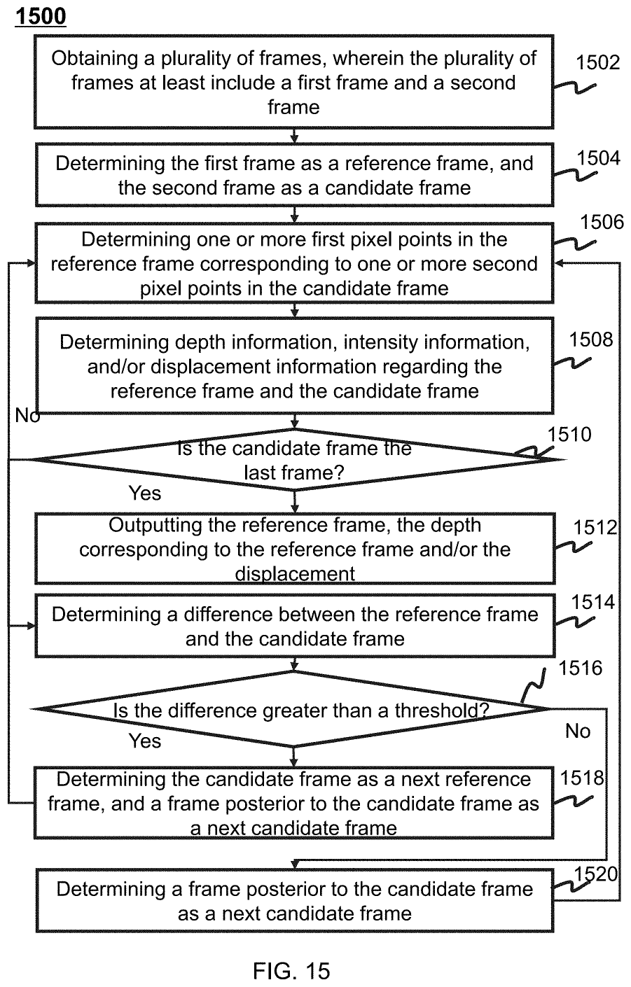

In 1420, the analysis module 310 may determine one or more reference frames based on the image data. In some embodiments, the image data may include the plurality of frames, the initial depth and/or the displacement of each pixel point in the each frame. In some embodiments, the analysis module 310 may select the one or more reference frames from the plurality of frames. More descriptions about of the reference frame may be found elsewhere in the present disclosure (e.g., FIG. 15, and the descriptions thereof). In some embodiments, the reference frames may be used to construct a map.

In 1430, the analysis module 310 may determine depth information and displacement information based on the one or more reference frames. In order to obtain the displacement information and the depth information of each frame, the analysis module 310 may process the image data. More descriptions of the determination of the displacement information and the depth information may be found elsewhere in the present disclosure (e.g., FIG. 4 and the descriptions thereof).

In 1440, the analysis module 310 may generate the map based on the one or more reference frames, the depth information and the displacement information. In some embodiments, the three-dimensional map may be obtained by combining the one or more reference frames and the corresponding displacements.

The map may be determined according to the plurality of frames and corresponding displacement information and depth information. In some embodiments, operation 1420 and operation 1430 may be performed reversely or simultaneously. For example, the process for determining the displacement information and the depth information in operation 1420 may further include determining the one or more reference frames in operation 1430. Operation 1430 may be a sub-operation of operation 1420 for determining the one or more reference frames. As described in connection with FIG. 4, the image data may be processed to generate one or more results. In some embodiments, the one or more results may include the displacement information (e.g., a camera displacement between two adjacent frames) and the depth information (e.g., a depth of an object in the two adjacent frames). In some embodiments, the one or more results may be adjusted according to the g2o closed loop detection technique. The adjusted displacement information may be generated. In some embodiments, the adjusted displacement may be used to generate the map. The analysis module 310 may generate the map based on the one or more reference frames and corresponding displacement information and depth information.

FIG. 15 is a flowchart illustrating an exemplary process for determining one or more reference frames according to some embodiments of the present disclosure. This process may be performed by the analysis module 310, the displacement determination unit 420, and the depth determination unit 430 based on image data obtained by the imaging sensor 810. Specifically, the analysis module 310 may determine the one or more reference frames based on the one or more results (e.g., the displacement information and the depth information).

In 1502, the analysis module 310 may obtain image data including a plurality of frames. The plurality of frames may at least include a first frame and a second frame. In some embodiments, the first frame may be a given frame, and the second frame may be a subsequent frame of the first frame. The imaging sensor 810 may acquire the first frame at a time point and acquire the second frame at a next time point. The plurality of frames may be sequential in a time domain.

In 1504, the analysis module 310 may determine the first frame as a reference frame and the second frame as a candidate frame.

In 1506, the analysis module 310 may determine one or more first pixel points in the reference frame corresponding to one or more second pixel points in the candidate frame. In some embodiments, the reference frame and the candidate frame may include an overlapping region. In this case, the first pixel point and the second pixel point may indicate a same position of an object in the overlapping region of the reference frame and the candidate frame. In some embodiments, the one or more first pixel points may be a set of pixel points Ω as described in FIG. 4. In some embodiments, the reference frame and the candidate frame may not include the overlapping region. Any region in the reference frame may not correspond to a region in the candidate frame. In this case, the pixel points in the reference frame and the candidate frame may not be selected as the first pixel points and/or the second pixel points.

In 1508, the analysis module 310 may determine the depth information, the intensity information, and/or the displacement information regarding the reference frame and the candidate frame. More descriptions of the determination of the depth information, the intensity information, and/or the displacement information may be found in the descriptions of FIG. 4.

In 1510, the analysis module 310 may determine whether the candidate frame is the last frame. The analysis module 310 may determine whether a next frame of the candidate frame exists in the time domain. If the candidate frame has the next frame, the process may proceed to operation 1512. Otherwise, the process may proceed to operation 1514.

In 1512, if the next frame of the candidate frame is the last frame, the analysis module 310 may output the reference frame, the depth corresponding to the reference frame and/or the displacement.

In 1514, the analysis module 310 may determine a difference between the reference frame and the candidate frame. In some embodiments, the difference between the reference frame and the candidate frame may be determined based on the intensity information of the reference frame and the candidate frame. In some embodiments, the intensity of the reference frame may be determined by the RGB intensity of the one or more first pixel points. The intensity of the candidate frame may be determined by the RGB intensity of the one or more second pixel points. In some embodiments, the intensity information of the reference frame and the candidate frame may be determined by performing operation 1504. In some embodiments, the operation for determining the intensity information of the reference frame and the candidate frame in operation 1514 may be performed before the operation for determining the difference between the reference frame and the candidate frame.

In 1516, the analysis module 310 may determine whether the difference between the reference frame and the candidate frame is greater than a threshold. If the difference between the reference frame and the candidate frame is greater than the threshold, the process may proceed to operation 1518. Otherwise, the process may proceed to operation 1520.

In 1518, if the difference between the reference frame and the candidate frame is greater than the threshold, the analysis module 310 may determine the candidate frame as a updated reference frame, and the frame posterior to the candidate frame as a updated candidate frame. In some embodiments, the frame posterior to the candidate frame may be adjacent to the candidate frame. The updated reference frame and the updated candidate frame may be sent to operation 1506. The process 1500 may be repeated.

In 1520, if the difference between the reference frame and the candidate frame is not greater than the threshold, the analysis module 310 may determine the frame posterior to the candidate frame as the updated candidate frame. The updated reference frame and the updated candidate frame may be sent to operation 1506. The process 1500 may be repeated.

In some embodiments, the analysis module 310 may process the update reference frame and the update candidate frame outputted in operation 1518 or operation 1520. In some embodiments, when the difference between the reference frame and the candidate frame is greater than the threshold, the update reference frame may be obtained by replacing the reference frame with the candidate frame. In some embodiments, the update candidate frame may be obtained by replacing the candidate frame with the next frame of the candidate frame. The replacement of the candidate frame may be unconditional, and the replacement of the reference frame may be conditional.

When the map is obtained in operation 1512, the process 1500 may be terminated. In some embodiments, a plurality of termination conditions may be predetermined in order to terminate the process 1500 in time. For example, a counter may be used in process 1500 to ensure the number of iterations of the process 1500 is not greater than a predetermined threshold.

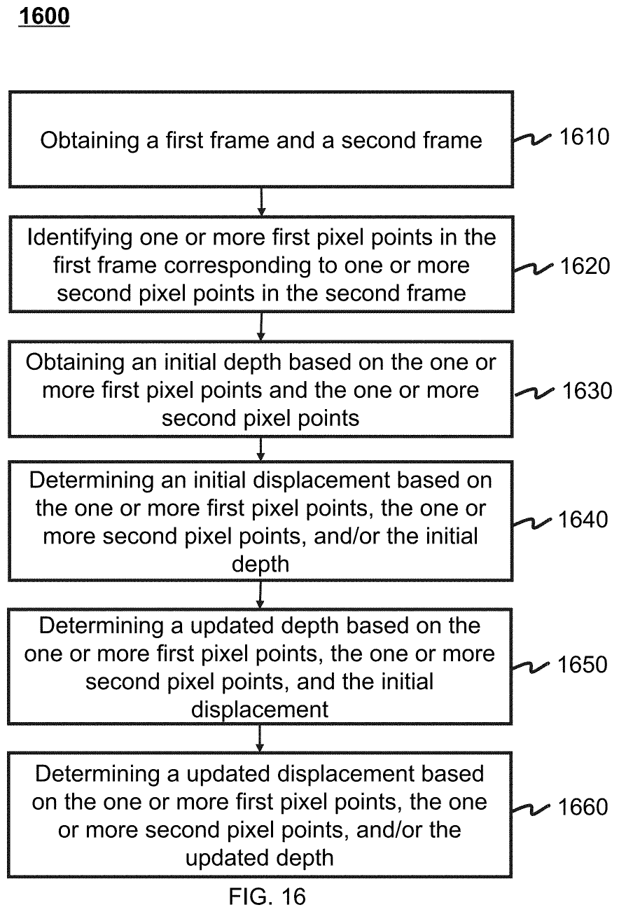

FIG. 16 is a flowchart illustrating an exemplary process for determining depth information regarding a reference frame and/or a candidate frame and displacement information according to some embodiments of the present disclosure. In some embodiments, the process may be performed by the analysis module 310. In some embodiments, the process may be similar to the process 1400 for obtaining the displacement and the depth of the frame as described in FIG. 4.

In 1610, the analysis module 310 may obtain a first frame and a second frame from a plurality of frames obtained by the imaging sensor 810. In some embodiments, the analysis module 310 may select the first frame and the second frame among the plurality of frames acquired by an imaging sensor. In some embodiments, the first frame and the second frame may be adjacent to each other in a time domain. The first frame may be a given frame, and the second frame may be a continuous frame relative to the first frame.

In 1620, the analysis module 310 may identify one or more first pixel points in the first frame corresponding to one or more second pixel points in the second frame. The pixel points in the first frame corresponding to the pixel points in the second frame may be identified by performing operation 1506 as described in connection with FIG. 15.

In 1630, the analysis module 310 may obtain an initial depth based on the one or more first pixel points and the one or more second pixel points. In some embodiments, the initial depth may be set as a zero matrix. In 1640, the analysis module 310 may determine an initial displacement based on the one or more first pixel points, the one or more second pixel points, and/or the initial depth. For example, operation 1640 may be implemented according to the Equation (1) described in FIG. 4.

In 1650, the analysis module 310 may determine a updated depth based on the one or more first pixel points, the one or more second pixel points, and the initial displacement. In some embodiments, operation 1650 may be implemented according to the Equation (2) described in FIG. 4.

In 1660, the analysis module 310 may determine a updated displacement based on the one or more first pixel points, the one or more second pixel points, and/or the updated depth. In some embodiments, operation 1660 may be implemented according to the Equation (1) described in FIG. 4. The initial depth may be replaced by the updated depth.

As described in connection FIG. 4, the initial displacement may be obtained firstly in order to determine the displacement according to the Equation (1). As illustrated in Equation (1), the initial displacement may be determined based on an initial value of the displacement. FIG. 17A is a flowchart illustrating an exemplary process for determining an initial value of a displacement according to some embodiments of the present disclosure. This process may be performed by the analysis module 310 based on image data obtained by the imaging sensor 810.

In 1710, the analysis module 310 may obtain image data. In some embodiments, an initial value of the displacement may be determined based on the image data. Specifically, the initial value of the displacement may be determined based on the displacement included in the image data. In some embodiments, the displacement in the image data may include the displacement of the motion unit (e.g., two wheels) and the displacement of the camera relative to the motion unit during a time interval in which the two adjacent frames are taken.

In 1720, the analysis module 310 may obtain a first displacement associated with the motion unit based on the image data. In some embodiments, the first displacement associated with the motion unit may be a displacement of a center of two wheels over a time period. In some embodiments, the first displacement associated with the motion unit may be a displacement of a point within the time period. The point may be coupled to a navigation sensor. In some embodiments, the navigation sensor may be located at the center of the two wheels. In some embodiments, the time period may be a time interval when the imaging sensor 810 acquires the two frames.

In 1730, the analysis module 310 may obtain a second displacement associated with the imaging sensor 810 relative to the motion unit. In some embodiments, the second displacement may be the relative displacement of the imaging sensor 810 relative to the motion unit. In some embodiments, the imaging sensor 810 may be a camera.

In 1740, the analysis module 310 may determine a third displacement associated with the imaging sensor 810 based on the first displacement and the second displacement. In some embodiments, the third displacement may be a sum of the vectors of the first displacement and the second displacement. In some embodiments, the value of the initial displacement may be determined based on the third displacement.

During the movement of the intelligent robot 110, a precise pose of the intelligent robot 110 may be determined by controlling the tripod head. In some embodiments, the pose of the intelligent robot 110 may be controlled by controlling a rotation angle of a shaft in the tripod head 930. FIG. 17B is a flowchart illustrating an exemplary process for determining a pose of the intelligent robot 110 according to some embodiments of the present disclosure. The process may be performed by the analysis module 310 based on a rotation angle of an axis of the tripod head 930.

In 1715, the analysis module 310 may obtain the image data. As described in connection with FIG. 17A, the image data may include the frame, the displacement, and the initial depth. In some embodiments, the image data may further include rotation information.

In 1725, the analysis module 310 may obtain a first rotation angle relative to a reference axis. The first rotation angle may relate to the motion unit based on the image data. In some embodiments, the first rotation angle of the reference axis regarding the motion unit may be obtained based on the rotation information in the image data. In some embodiments, the first rotation angle may be an angle rotated within a time period. In some embodiments, the time period may be a time interval between the two time points at which the imaging sensor 810 acquires the two frames.

In 1735, the analysis module 310 may obtain a second rotation angle relative to the motion unit over a time period. The motion unit may relate to the imaging sensor. In some embodiments, the second rotation angle may be a relative rotation angle of the imaging sensor 810 relative to the motion unit. In some embodiments, the imaging sensor 810 may be a camera.

In 1745, the analysis module 310 may determine a third rotation angle relative to the reference axis. The reference axis may relate to the imaging sensor 810. In some embodiments, the third rotation angle may be determined based on the first rotation angle and the second rotation angle. In some embodiments, the third rotation angle may be a sum of the vectors of the first rotation angle and the second rotation angle.

During the movement of the intelligent robot 110, the motion assembly 820 and the tripod head 930 may be configured with the sensor(s) 230 to obtain the information. In some embodiments, the sensor(s) 230 may be disposed in the carrier 1010 or a smart phone supported by the tripod head 930. In some embodiments, the motion assembly 920 and the tripod head 930 may need to be kept stable in order to obtain accurate and reliable information. More description about keeping balance between the motion assembly 920 and the tripod head 930 in a horizontal plane may be found in FIG. 18, and the descriptions thereof.

FIG. 18 is a block diagram illustrating an exemplary scheme for determining an included angle between a horizontal plane and a Z axis by a gyroscope and an accelerometer according to some embodiments of the present disclosure. In some embodiments, the horizontal plane may be a mounting surface of the carrier 1010. The included angle between the horizontal plane and the Z axis may be determined based on the gyroscope data and the accelerometer data. In some embodiments, the horizontal plane may be a relative plane that the tripod head 930 detects a pitch angle of the tripod head 930.

As shown in FIG. 18, the scheme may include an adder 1810, an integrator 1820, a component extractor 1830, and an adder 1840. The adder 1810, the integrator 1820, the component extractor 1830, and the adder 1840 may form a feedback loop for determining an output angle. The integrator 1820 may obtain the included angle between the horizontal plane and the Z axis in each frame obtained by the imaging sensor 810. It is assumed that the imaging sensor 810 obtains the first frame at a time point t1, and obtains the second frame at a time point t2. The gyroscope 830 and the accelerometer 820 may obtain an angular velocity and included angle information at the time pints t1 and t2. In some embodiments, a feedback output angle θ₁ regarding the first frame obtained at the time point t1, the gyroscope data and the accelerometer data obtained at the time point t2 may be used to determine an output angle θ₂ regarding the second frame obtained at the time point t2.

The gyroscope data and the accelerometer data regarding the first frame may be processed at the time point t1. The integrator 1820 may generate an output angle θ₁ regarding the first frame. The accelerometer 820 may generate a first included angle θ₁′. The adder 1840 may generate a second included angle θ₁″ based on the output angle θ₁ and the first included angle θ₁′. In some embodiments, the second included angle θ₁″ may be determined according to the subtraction of vectors of the output angle θ₁ and the first included angle θ₁′. The component extractor 1830 may determine a compensation angular velocity ω₁″ based on the second included angle θ₁″. In some embodiments, the component extractor 1830 may be a differentiator.

The gyroscope data and the accelerometer data of the second frame may be processed at the time point t2. The gyroscope 830 may generate an angular velocity ω₂. The adder 1810 may generate a corrected angular velocity ω₂′ based on the angular velocity ω₂ and a compensated angular velocity ω₁″. In some embodiments, the corrected angular velocity ω₂′ may be determined by adding vectors of the angular velocity ω₂ and the compensation angular velocity ω₁″. The integrator 1820 may output an included angle θ₂ regarding the second frame at the time point t2 based on the corrected angular velocity ω₂′.

In some embodiments, the method described in FIG. 18 may be performed by the processor 210. For example, the gyroscope data and the accelerometer data may be transmitted to the processor 210 via an API interface (e.g., a part of a smartphone). When each frame is obtained, the processor 210 may determine the output angle. In some embodiments, the included angle between the horizontal plane and the Z axis may be detected when each frame is obtained. The balance may be maintained based on the real-time output angle regarding the each frame.

FIG. 19 is a flowchart illustrating an exemplary process 1900 for determining an angle regarding a frame. The process 1900 may be performed by the processor 210.

In 1910, the processor 210 may obtain a plurality of frames including a first frame and a second frame. In some embodiments, the first frame and the second frame may be acquired by the imaging sensor 810 at different time intervals. For example, the imaging sensor 810 may acquire a first frame at a time point t₁, and acquire a second frame and at a time point t₂. The time interval between the time point t₁ and the time point t₂ may be a sampling interval of the imaging sensor 810.

In 1920, the processor 210 may obtain gyroscope data and accelerometer data regarding the first frame and/or the second frame. In some embodiments, the gyroscope data and the accelerometer data may include parameters, such as an angular velocity and an angle.

In 1930, the processor 210 may determine first angle information based on the accelerometer data regarding the first frame. In some embodiments, the first angle information may include a first angle.

In 1940, the processor 210 may determine compensation angle information based on the first angle information and the angle information regarding the first frame. In some embodiments, the angle information regarding the first frame may be an output angle regarding the first frame. In some embodiments, the first angle information may be determined by subtracting a vector by the output angle regarding the first frame. In some embodiments, the compensation angle information may be a compensation angle speed. The compensation angle speed may be determined by subtracting the output angle regarding the first frame from the first angle information by the component extractor 1830.

In 1950, the processor 210 may determine second angle information based on the compensation angle information and the gyroscope data regarding the second frame. In some embodiments, at the time point t₂, the second angle data may be the angle between the horizontal plane and the Z axis regarding the second frame determined by the processor 210.

As illustrated in connection with FIG. 18 and FIG. 19, the output angle regarding the second frame may be fed back to the output angle regarding the first frame. The processor 210 may obtain the output angle of each frame based on the gyroscope data and the accelerometer data in the loop. In some embodiments, if the included angle between the horizontal plane and the Z axis exceeds a certain threshold, a balance control signal may be generated.

The process for maintaining the horizontal balance of the motion assembly 920 or the tripod head 930 is illustrated in FIG. 18 and FIG. 19. During the movement of the intelligent robot 110, the sensor disposed in the smartphone supported by the tripod head 930 may obtain the information. In some embodiments, the information may include the image data, the gyroscope data, the accelerometer data, and the data obtained from other sensors. It is necessary to maintain the horizontal balance in order to the processor 210 may stably obtain the information from the second type sensor 1240 in the smartphone. In some cases, for the second type sensor 1240 in the smartphone supported by the tripod head 930, the information from the second type sensor 1240 may not be obtained stably due to the uneven road condition. In some embodiments, it is necessary to maintaining vertical balance so as that the sensor in the smartphone may detect the information stably.

FIG. 20 is a flowchart illustrating an exemplary process 2000 for adjusting a vertical displacement of a second type of sensor 1240 in a smartphone according to some embodiments of the present disclosure. In some embodiments, the process may be performed by the processor 210. The dynamic Z buffer bar 1120 illustrated in FIG. 11 may be controlled based on the control parameters generated by the intelligent robot control module 330.

In 2010, the processor 210 may obtain a first displacement of a motor along a rotation axis. In some embodiments, the rotation axis may be a Z axis. The first displacement may be represented by a vector along the Z axis.

In 2020, the processor 210 may determine whether the displacement of the motor along the Z axis is greater than a threshold. In some embodiments, the threshold may be an extreme value. If the displacement is within the extreme value, the second type of sensor 1240 may detect stability information.

In 2030, in response to a determination that the displacement of the motor is greater than the threshold, the processor 210 may generate a first control signal to move the motor to an initial position. In some embodiments, the initial position may be a predetermined position for obtaining stability information.

In 2040, the processor 210 may output the first control signal to the motor to direct the second type of sensor 1240 disposed in the smartphone to return to the initial position so as to detect the stability information.

In 2050, in response to a determination that the displacement of the motor is not greater than the threshold, the processor 210 may obtain a first acceleration along the rotation axis. In some embodiments, the acceleration may be obtained by the accelerometer 820 disposed in the smartphone.

In 2060, the processor 210 may generate a second acceleration based on the first acceleration. In some embodiments, the second acceleration may be a filtered first acceleration.

In 2070, the processor 210 may determine a second displacement based on the second acceleration. In some embodiments, the second displacement may be calculated based on an integrated value of the second acceleration. In some embodiments, the second displacement may be represented by a vector along the Z axis.

In 2080, the processor 210 may generate a second control signal based on the second displacement to control a movement of the motor. In some embodiments, the second control signal may determine a gap of the displacement (e.g., an available movement range) between the second displacement and the threshold. The processor 210 may control the sensor in the smartphone to move along the Z axis.

In 2090, the sensor 210 may output the second control signal to the motor.

The present disclosure is described and illustrated using a plurality of embodiments and it will be appreciated that those skilled in the art may make various modifications in form and detail without departing from the spirit and scope of the present disclosure as defined in the appended claims and their equivalent description.

1. A system for physiological monitoring, comprising:
sensors, wherein the sensors include a motion sensor and a medical monitoring sensor, the motion sensor includes a first type of sensor and a second type of sensor, and the medical monitoring sensor is configured to acquire physiological information of a user;
a motion assembly including a wheel, a carrier, and the first type of sensor;
a tripod head including the second type of sensor;
a processor, including an analysis module, a navigation module, and a control module, configured to:
establish a communication with the tripod head and the motion assembly, respectively;
obtain information from one or more of the first type sensor and the second type sensor respectively;
determine a destination and a location of the system;
construct a map based on the information;
plan a route for the system based on the map;
determine control parameters for the system based on the route and the information; and
control a movement and a pose of the system based on the control parameters.

2. The system of claim 1, wherein the medical monitoring sensor includes at least one of a blood pressure measuring device, an ECG monitoring device, a blood measuring device, a pulse wave detector, a brain wave monitor, a heart rate detector, a pulse oximeter, a blood oxygen detector, a respiratory detector, an invasive blood pressure detector, a non-invasive blood pressure detector, a cardiac output detector, a body temperature detector, or a blood gas sensor.

3. The system of claim 1, wherein the medical monitoring sensor is configured to acquire the physiological information of the user in real time.

4. The system of claim 1, wherein the medical monitoring sensor is in contact with the body surface of the user.

5. The system of claim 1, wherein the physiological information includes at least one of electrocardiographic information, heart rate information, pulse information, blood pressure information, blood oxygenation information, respiratory information, invasive blood pressure information, non-invasive blood pressure information, cardiac output, body temperature information, or blood gas information.

6. The system of claim 1, wherein the processor is further configured to compare the physiological information with a predetermined safety threshold, and determine a warning signal when an abnormality occurs.

7. The system of claim 6, wherein the predetermined safety threshold includes at least one of a safe blood pressure value, a safe blood oxygen level value, a safe heart rate value, or a safe pulse rate value.

8. The system of claim 6, wherein the processor is further configured to send the warning signal to a smart device to notify a user.

9. The system of claim 1, wherein the processor is further configured to:
predict future physiological information based on historical physiological information;
compare the future physiological information with the predetermined safety threshold, and determine an early warning signal when the abnormality occurs.

10. A method for physiological monitoring, comprising:
establishing a communication with a tripod head and a motion assembly, respectively;
obtaining information from sensors, wherein the sensors include a motion sensor including a first type of sensor and a second type of sensor, and a medical monitoring sensor configured to acquire physiological information of a user;
determining a destination and a location of an intelligent robot based on the obtained information or part of the obtained information;
constructing a map based on the destination and the location of the intelligent robot;
planning a route from the location of the intelligent robot based on the map;
determining control parameters for the intelligent robot based on the route and the obtained information; and
controlling a movement and a pose of the intelligent robot based on the control parameters.

11. The method of claim 10, wherein the medical monitoring sensor includes at least one of a blood pressure measuring device, an ECG monitoring device, a blood measuring device, a pulse wave detector, a brain wave monitor, a heart rate detector, a pulse oximeter, a blood oxygen detector, a respiratory detector, an invasive blood pressure detector, a non-invasive blood pressure detector, a cardiac output detector, a body temperature detector, or a blood gas sensor.

12. The method of claim 10, wherein the medical monitoring sensor is configured to acquire the physiological information of the user in real time.

13. The method of claim 10, wherein the medical monitoring sensor is in contact with the body surface of the user.

14. The method of claim 10, wherein the physiological information includes at least one of electrocardiographic information, heart rate information, pulse information, blood pressure information, blood oxygenation information, respiratory information, invasive blood pressure information, non-invasive blood pressure information, cardiac output, body temperature information, or blood gas information.

15. The method of claim 10, further comprising:
comparing the physiological information with a predetermined safety threshold; and
determining a warning signal when an abnormality occurs.

16. The method of claim 15, wherein the predetermined safety threshold includes at least one of a safe blood pressure value, a safe blood oxygen level value, a safe heart rate value, or a safe pulse rate value.

17. The method of claim 15, further comprising:
sending the warning signal to a smart device to notify the user.

18. The method of claim 10, further comprising:
predicting future physiological information based on historical physiological information;
comparing the future physiological information with the predetermined safety threshold, and determine an early warning signal when the abnormality occurs.

19. A non-transitory computer readable medium, comprising at least one set of instructions for physiological monitoring, wherein when executed by at least one processor of a computing device, the at least one set of instructions causes the computing device to perform a method, the method comprising:
establishing a communication with a tripod head and a motion assembly, respectively;
obtaining information from sensors, wherein the sensors include a motion sensor including a first type of sensor and a second type of sensor, and a more medical monitoring sensor configured to acquire physiological information of a user;
determining a destination and a location of an intelligent robot based on the obtained information;
constructing a map based on the destination and the location of the intelligent robot;
planning a route from the location of the intelligent robot based on the map;
determining control parameters for the intelligent robot based on the route and the obtained information; and
controlling a movement and a pose of the intelligent robot based on the control parameters.

20. The non-transitory computer readable medium of claim 19, wherein the physiological information includes at least one of electrocardiographic information, heart rate information, pulse information, blood pressure information, blood oxygenation information, respiratory information, invasive blood pressure information, non-invasive blood pressure information, cardiac output, body temperature information, or blood gas information.
